SEQUENCE LISTING (5 sequences)

SEQ ID NO: 1 - protein, 304 aa, Homo sapiens
MIYTMKKVHALWASVCLLLNLAPAPLNADSEEDEEHTIITDTELPPLKLMHSFCAFKADDGPCKAIMKRFFFNIFTRQCEEFIYGGCEGNQNRFESLEECKKMCTRDNANRIIKTTLQQEKPDFCFLEEDPGICRGYITRYFYNNQTKQCERFKYGGCLGNMNNFETLEECKNICEDGPNGFQVDNYGTQLNAVNNSLTPQSTKVPSLFEFHGPSWCLTPADRGLCRANENRFYYNSVIGKCRPFKYSGCGGNENNFTSKQECLRACKKGFIQRISKGGLIKTKRKRKKQRVKIAYEEIFVKNM

SEQ ID NO: 2 - protein, 58 aa, Bos taurus [sequence omitted]

SEQ ID NO: 3 - protein, 56 aa, Artificial Sequence
  Synthetically generated peptide
EACNLPIVRGPCIAFFPRWAFDAVKGKCVLFPYGGCQGNGNKFYSEKECREYCGVP

SEQ ID NO: 4 - protein, 267 aa, Homo sapiens
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 5 - DNA, 920 bp, Homo sapiens
gcacggaggggcagagaccccggagccccagccccaccatgaccctcggccgccgactcgcgtgtcttttcctcgcctgtgtcctgccggccttgctgctggggggcaccgcgctggcctcggagattgtggggggccggcgagcgcggccccacgcgtggcccttcatggtgtccctgcagctgcgcggaggccacttctgcggcgccaccctgattgcgcccaacttcgtcatgtcggccgcgcactgcgtggcgaatgtaaacgtccgcgcggtgcgggtggtcctgggagcccataacctctcgcggcgggagcccacccggcaggtgttcgccgtgcagcgcatcttcgaaaacggctacgaccccgtaaacttgctcaacgacatcgtgattctccagctcaacgggtcggccaccatcaacgccaacgtgcaggtggcccagctgccggctcagggacgccgcctgggcaacggggtgcagtgcctggccatgggctggggccttctgggcaggaaccgtgggatcgccagcgtcctgcaggagctcaacgtgacggtggtgacgtccctctgccgtcgcagcaacgtctgcactctcgtgaggggccggcaggccggcgtctgtttcggggactccggcagccccttggtctgcaacgggctaatccacggaattgcctccttcgtccggggaggctgcgcctcagggctctaccccgatgcctttgccccggtggcacagtttgtaaactggatcgactctatcatccaacgctccgaggacaacccctgtccccacccccgggacccggacccggccagcaggacccactgagaagggctgcccgggtcacctcagctgcccacacccacactctccagcatctggcacaataaacattctctgttttgt

(19) United States
(12) Patent Application Publication
(10) Pub. No.: US 2007/0020252 A1
(43) Pub. Date: Jan. 25, 2007

(54) Modified protease inhibitors

(76) Inventors: Robert C. Ladner, Ijamsville,, MD (US); Aaron K. Sato, Somerville, MA (US); Arthur C. Ley, Newton, MA (US); Michelle Amaral, Birmingham, AL (US); Mary J. Bossard, Madison, AL (US); Michael J. Roberts, Williamburg, VA (US); Yan Zhang, Madison, AL (US)

Correspondence Address: FISH & RICHARDSON PC, P.O. BOX 1022, MINNEAPOLIS, MN, 55440-1022, US

(21) Appl. No.: 10/570,186

(22) PCT Filed: Aug. 30, 2004
(86) PCT No.: PCT/US04/28256

§ 371 Date: Aug. 28, 2006

Related U.S. Application Data
(60) Provisional application No. 60/498,845, filed on Aug. 29, 2003.

(51) Int. Cl. A61K 38/54; C12N 9/99

(52) U.S. Cl. 424/94.2; 435/184

(57) ABSTRACT

DX-890 inhibits elastase. DX-890 can be attached a single polyethylene glycol moiety. The polyethylene glycol is at least 18 kDa in molecular weight and is attached to the polypeptide by a single covalent bond to the N-terminus of the polypeptide.

### CROSS-REFERENCE TO RELATED APPLICATIONS

This application claims priority to U.S. Application Ser. No. 60/498,845, filed on Aug. 29, 2003.

### BACKGROUND

The invention relates to modified protease inhibitors.

### SUMMARY

In one aspect, the invention features a compound that include: a) a polypeptide including a Kunitz domain that specifically binds and inhibits an elastase (e.g., human neutrophil elastase (hNE)); and b) a non-protein moiety that is physically associated with the polypeptide and increases the molecular weight of the compound, wherein the non-protein moiety has a molecular weight of at least 7 kDa and the compound has a molecular weight of greater than 18 kDa.

In one embodiment, the non-protein moiety includes a hydrophilic polymer, e.g., a substantially homogeneous polymer. The polymer can be branched or unbranched. For example, the polymer has a molecular weight of at least 10, 18, 20, 28, or 30 kDa. In one embodiment, the polymer is a polyalkylene oxide. For example, at least 20, 30, 50, 70, 80, 90, or 95% of the copolymer blocks of the polymer are ethylene glycol. In one embodiment, the polymer is polyethylene glycol.

In one embodiment, the compound has the following structure:
P—X⁰—[(CR′R″)ₙ—X¹]ₐ—(CH₂)ₘ—X²—R^{t}

wherein P is the polypeptide,

each of R′ and R″ is, independently, H, or C₁-C₁₂ alkyl;

X⁰ is O, N—R¹, S, or absent, wherein R¹ is H, C₁-C₁₂ alkyl or aryl,

X¹ is O, N—R², S, wherein R² is H, alkyl or aryl,

X² is O, N—R³, S, or absent, wherein R³ is H, alkyl or aryl,

each n is between 1 and 5, e.g., 2,

a is at least 4,

m is between 0 and 5, and

R^{t} is H, C₁-C₁₂ alkyl or aryl.

R′ and R″ can be H. In one embodiment, R′ or R″ is independently, H, or C1-C4, C1-C6, or C1-C10 alkyl.

In one embodiment, the compound has the following structure:
P—X⁰—[CH₂CH₂O]ₐ—(CH₂)ₘ—X²—R^{t}

wherein P is the polypeptide,

a is at least 4,

m is between 0 and 5,

X² is O, N—R¹, S, or absent, wherein R¹ is H, alkyl or aryl,

X⁰ is O, N—R², S, or absent, wherein R² is H, alkyl or aryl, and

R^{t} is H, C₁-C₁₂ alkyl or aryl. For example, X² is O, and R^{t} is H.

In one embodiment, the polypeptide is less than 14, 8, or 7 kDa in molecular weight. In one embodiment, the compound includes only a single Kunitz domain.

In one embodiment, the Kunitz domain includes the amino acid sequence of DX-890 or an amino acid sequence that differs by at least one, but fewer than six, five, four, three, or two amino acid differences (e.g., substitutions, insertions, or deletions) from the amino acid sequence of DX-890. Typically, the Kunitz domain does not naturally occur in humans. The Kunitz domain may include an amino acid sequence that differs by fewer than ten, seven, or four amino acids from a human Kunitz domain.

In one embodiment, the Kᵢ of the compound is within a factor of 0.5 to 1.5, 0.8 to 1.2, or 0.3 to 3.0 of the Kᵢ of the unmodified polypeptide for elastase. For example, the Kᵢ for hNE can be less than 100, 50, 18, 12, 10, or 9 pM.

In one embodiment, the compound has a circulatory half life in a rabbit or mouse model that is at least 1.5, 2, 4, or 8 fold greater than a substantially identical compound that does not include the polymer. The compound can have a beta-phase circulatory half life in a rabbit or mouse model that has an amplitude at least 1.5, 2, 2.5, or 4 fold greater than a substantially identical compound that does not include the non-protein moiety. The compound can have an alpha-phase circulatory half life in a rabbit or mouse model that has an amplitude at least 20, 30, 40, or 50% smaller than a substantially identical compound that does not include the non-protein moiety. For example, the compound has a beta phase with an amplitude of at least 40, 50, 60, or 65%. In one embodiment, the compound has a beta phase circulatory half life in a mouse or rabbit model of at least 2, 3, 4, 5, 6, or 7 hours. In one embodiment, the compound has a beta phase circulatory half life in a 70 kg human of at least 6 hours, 12 hours, 24 hours, 2 days, 5 days, 7 days, or 10 days.

In one embodiment, the polypeptide is attached to a single molecule of the polymer. For example, the N-terminus of the polypeptide is attached to the polymer. In one embodiment, the polyethylene glycol is attached by coupling monomethoxy-PEG propionaldehyde or monomethoxy-PEG succinimidyl propionic acid to the polypeptide. The compound can formed by coupling of MPEG at about pH 6.8 to 8.0, or pH 7.2 to 7.6, e.g., about pH 7.4 or about pH 5.6 to 6.5, e.g., 5.8 to 6.2, e.g., about pH 6.

In another aspect, the invention features a compound that includes (1) a polypeptide including the amino acid sequence of DX-890 or an amino acid sequence that differs by at least one, but fewer than six, five, four, three, or two amino acid differences (e.g., substitutions, insertions, or deletions) from the amino acid sequence of DX-890, and (2) polyethylene glycol wherein the polyethylene glycol is at least 15, 18, 20, 25, 27, or 30 kDa in molecular weight and is attached to the polypeptide by a single covalent bond. In one embodiment, the polyethylene glycol is attached to the N-terminus.

In one embodiment, the amino acid sequence differs by at least one amino acid from the amino acid sequence of DX-890. The amino acid sequence is identical to the amino acid sequence of DX-890 at one or more positions (e.g., at least two, three, five, seven, ten, twelve, thirteen, fourteen, or all) corresponding to positions 5, 13, 14, 16, 17, 18, 19, 30, 31, 32, 34, 38, 39, 51, and 55 according to the BPTI numbering.

The invention also features a preparation that includes a compound described herein, e.g., above. For example, the compound is present at a concentration of at least 0.1, 1, 2, or 5 mg of polypeptide per milliliter, e.g., in a solution between pH 6-8. In one embodiment, the compound produces a major peak by size exclusion chromatography that includes at least 70% the compound relative to the injectate. In one embodiment, the molecular weight of 95% of the species of the compound are within 5, 4, 3, 2, or 1 kDa of the average molecular weight of the compound.

In another aspect, the invention features a pharmaceutical preparation that includes (1) a compound described herein, and (2) a pharmaceutically acceptable carrier. In one embodiment, at least 60, 70, 80, 85, 90, 95, 97, 98, 99, or 100% of the compounds in the preparation have an identical distribution of PEG molecules attached thereto. In one embodiment, the preparation is aqueous and the compound is present at a concentration of at least 0.1 mg of polypeptide per milliliter. In one embodiment, injection of the preparation into a mouse results in less than 50, 30, 25, 15, or 10% of the compound is an SEC peak with higher mobility than the preparation after 12 hours.

In another aspect, the invention features a substantially (e.g., at least 70, 75, 80, 85, 90, 95, or 100%) monodisperse preparation that includes a compound described herein. For example, the compound is present at a concentration of at least 0.05, 0.1, 0.2, 0.5, 0.8, 1.0, 1.5, 2.0, or 2.5 milligrams of polypeptide per milliliter or between 0.05 and 10 milligrams of polypeptide per milliliter. In one embodiment, the preparation is dry. For example, the preparation includes particles or is in the form of a powder.

In another aspect, the invention features an aqueous preparation that includes: a compound that includes an elastase-inhibiting Kunitz domain conjugated to a hydrophilic and substantially homogeneous polymer. In one embodiment, the Kunitz domain includes the amino acid sequence of DX-890 or an amino acid sequence that differs by at least one, but fewer than six, five, four, three, or two amino acid differences (e.g., substitutions, insertions, or deletions) from the amino acid sequence of DX-890. The invention also provides a sealed container that includes the preparation. The container can be opaque to light. The container can include printed information on an external region of the container.

In another aspect, the invention features a method that includes: providing a polypeptide that includes a Kunitz domain that inhibits elastase; contacting the polypeptide to a hydrophilic polymer (e.g., a polyalkylene oxide) that includes a single reactive group that can form a covalent bond with the polypeptide under conditions suitable for bond formation, thereby providing a modified elastase inhibitor.

In one embodiment, the hydrophilic polymer is mono-activated. For example, the hydrophilic polymer is alkoxy-terminated. In one embodiment, the polymer includes a succinimidyl group.

In one embodiment, the polymer is a polyethylene glycol, e.g., monomethoxy-polyethylene glycol. For example, the polymer is mPEG propionaldehyde or mPEG succinimidyl propionic acid.

In one embodiment, the conditions are between pH 5.5 and 6.5 or between pH 6.5 and 8.0. In one embodiment, the hydrophilic polymer is covalently attached to the N-terminus of the polypeptide.

The method can further include separating polypeptides that have a single attached polymer from other products and reactants. The method can further include chromatographically separating products of the contacting, e.g., using ion exchange chromatography or size exclusion chromatography.

In another aspect, the invention features a method for preparing a conjugate of DX-890, said method including: reacting DX-890 with an activated PEG reagent suitable for coupling to amino groups present on DX-890 under conditions effective to PEGylate one or more amino sites of said DX-890 to produce a conjugate described herien. In one embodiment, the activated PEG reagent is an electrophilically activated PEG. In one embodiment, the activated PEG reagent is selected from the group consisting of reactive esters of methoxy-PEG propionic acid, reactive esters of methoxy-PEG butanoic acid, activated esters of methoxy-PEG α-methyl substituted butanoate, activated esters of methoxy-PEG benzamide carbonate, and activated esters of methoxyPEG. For example, the reacting step is carried out in aqueous buffer, e.g., at a pH ranging from about 5.5 to about 7.6. The method can further include purifying the conjugate formed in said reacting step, e.g., using column chromatography, e.g., ion exchange chromatography. For example, the ion exchange chromatography is cation exchange chromatography using an aqueous eluant at a pH of less than about 6.0. The purification step can be effective to obtain a purified PEGylated DX-890 conjugate mixture, wherein all of the DX-890 molecules have the same number of PEG moieties covalently attached thereto.

The invention also features a modified elastase inhibitor prepared by a method described herein, e.g., the above methods.

In another aspect, the invention features a method of treating or preventing a pulmonary disorder. The method includes administering a compound described herein to a subject, e.g., in an amount effective to ameliorate at least one symptom of the disorder. For example, the compound includes a) a polypeptide including a Kunitz domain that specifically binds and inhibits an elastase (e.g., human neutrophil elastase (hNE)); and b) a non-protein moiety that is physically associated with the polypeptide and increases the molecular weight of the compound. For example, the compound includes (1) a polypeptide including the amino acid sequence of DX-890 or an amino acid sequence that differs by at least one, but fewer than six, five, four, three, or two amino acid differences (e.g., substitutions, insertions, or deletions) from the amino acid sequence of DX-890, and (2) polyethylene glycol wherein the polyethylene glycol is at least 15, 18, 20, 25, 27, or 30 kDa in molecular weight.

In one embodiment, the compound is administered no more than once every 12, 24, 36, or 72 hours. In another embodiment, the compound is administered no more than once every four, seven, ten, twelve, or fourteen days. The compound can be administered once or at multiple times (e.g., regularly).

In one embodiment, the administering includes pulmonary delivery. For example, the administering includes actuation of an inhaler and/or nebulization. In one embodiment, the administering includes delivery of the composition directly or indirectly into the circulatory system. For example, the administering includes injection or intravenous delivery.

In one embodiment, the subject has cystic fibrosis or a genetic defect in the cystic fibrosis gene. In another embodiment, the subject has chronic obstructive pulmonary disease.

The symptom can be lung tissue integrity or an index of tissue destruction.

In another aspect, the invention features a method of treating or preventing a inflammatory disorder. The method includes: administering a compound described herein to a subject, e.g., in an amount effective to ameliorate at least one symptom of the disorder. For example, the compound includes a) a polypeptide including a Kunitz domain that specifically binds and inhibits an elastase (e.g., human neutrophil elastase (hNE)); and b) a non-protein moiety that is physically associated with the polypeptide and increases the molecular weight of the compound. For example, the compound includes (1) a polypeptide including the amino acid sequence of DX-890 or an amino acid sequence that differs by at least one, but fewer than six, five, four, three, or two amino acid differences (e.g., substitutions, insertions, or deletions) from the amino acid sequence of DX-890, and (2) polyethylene glycol wherein the polyethylene glycol is at least 15, 18, 20, 25, 27, or 30 kDa in molecular weight.

In one embodiment, the disorder is an inflammatory bowel disorder, e.g., Crohn's disease or ulcerative colitis. In one embodiment, the compound is delivered by a suppository.

In one embodiment, the compound is administered no more than once every 12, 24, 36, or 72 hours. In another embodiment, the compound is administered no more than once every four, seven, ten, twelve, or fourteen days. The compound can be administered once or at multiple times (e.g., regularly).

In another aspect, the invention features a method of treating or preventing a disorder characterized at least in part by inappropriate elastase activity or neutrophil activity. The method includes administering a compound described herien to a subject, e.g., in an amount effective to ameliorate at least one symptom of the disorder or to alter elastase or neutrophil activity, e.g., to reduce elastase-mediated proteolysis. For example, the disorder is rheumatoid arthritis.

In one embodiment, the compound is administered no more than once every 12, 24, 36, or 72 hours. In another embodiment, the compound is administered no more than once every four, seven, ten, twelve, or fourteen days. The compound can be administered once or at multiple times (e.g., regularly).

Many of the examples provided herein describe methods and compositions that relate to Kunitz domains and a particular protease target—elastase. However, these methods and compositions can be modified to provide corresponding methods and compositions that relate to other targets, e.g., other proteases or other proteins. Similarly they can be modified to corresponding methods and compositions that relate to polypeptides that do not include a Kunitz domain or that include a Kunitz domain and other types of domains.

As used herein, “binding affinity” refers to the apparent association constant or Ka. The Ka is the reciprocal of the dissociation constant (Kd). A ligand may, for example, have a binding affinity of at least 10⁵, 10⁶, 10⁷, 10⁸, 10⁹, 10¹⁰, 10¹¹, or 10¹² M⁻¹ for a particular target molecule. Higher affinity binding of a ligand to a first target relative to a second target can be indicated by a higher Ka (or a smaller numerical value Kd) for binding the first target than the Ka (or numerical value Kd) for binding the second target. In such cases the ligand has specificity for the first target relative to the second target. Ka measurements for binding to hNE are typically made under the following conditions: 50 mM HEPES, pH 7.5, 150 mM NaCl, and 0.1% Triton X-100 at 30° C. using 100 pM of the hNE.

Binding affinity can be determined by a variety of methods including equilibrium dialysis, equilibrium binding, gel filtration, ELISA, surface plasmon resonance, or spectroscopy (e.g., using a fluorescence assay). These techniques can be used to measure the concentration of bound and free ligand as a function of ligand (or target) concentration. The concentration of bound ligand ([Bound]) is related to the concentration of free ligand ([Free]) and the concentration of binding sites for the ligand on the target where (N) is the number of binding sites per target molecule by the following equation:
[Bound]=N·[Free]/((1/Ka)+[Free])

It is not always necessary to make an exact determination of Ka, though, since sometimes it is sufficient to obtain a quantitative measurement of affinity, e.g., determined using a method such as ELISA or FACS analysis, is proportional to Ka, and thus can be used for comparisons, such as determining whether a higher affinity is, e.g., 2 fold higher.

An “isolated composition” refers to a composition that is removed from at least 90% of at least one component of a natural sample from which the isolated composition can be obtained. Compositions produced artificially or naturally can be “compositions of at least” a certain degree of purity if the species or population of species of interests is at least 5, 10, 25, 50, 75, 80, 90, 95, 98, or 99% pure on a weight-weight basis.

An “epitope” refers to the site on a target compound that is bound by a ligand, e.g., a polypeptide ligand such as a Kunitz domain, small peptide, or antibody. In the case where the target compound is a protein, for example, an epitope may refer to the amino acids that are bound by the ligand. Such amino acids may be contiguous or non-contiguous with respect to the underlying polypeptide backbone. Overlapping epitopes include at least one common amino acid residue.

As used herein, the term “substantially identical” (or “substantially homologous”) is used herein to refer to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities. In the case of Kunitz domains, the second domain has the same specificity and has at least 50% of the affinity of the first domain. A sufficient degree of identity may be about 85%, 90%, 91%, 92%, 93%, 94%, 95%, 96%, 97%, 98%, 99% or higher.

Sequences similar or homologous (e.g., at least about 85% sequence identity) to the sequences disclosed herein are also part of this application. In some embodiment, the sequence identity can be about 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., highly stringent hybridization conditions), to the complement of the strand. The nucleic acids may be present in whole cells, in a cell lysate, or in a partially purified or substantially pure form.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

As used herein, the term “homologous” is synonymous with “similarity” and means that a sequence of interest differs from a reference sequence by the presence of one or more amino acid substitutions (although modest amino acid insertions or deletions) may also be present. Presently preferred means of calculating degrees of homology or similarity to a reference sequence are through the use of BLAST algorithms (available from the National Center of Biotechnology Information (NCBI), National Institutes of Health, Bethesda Md.), in each case, using the algorithm default or recommended parameters for determining significance of calculated sequence relatedness. The percent identity between two amino acid or nucleotide sequences can also be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified. Accordingly, nucleic acids that hybridize with appropriate stringency to nucleic acids that encode a polypeptide described herein are provided as are polypeptides that are encode by such nucleic acids.

It is understood that a polypeptide described herein (e.g., a polypeptide that includes a Kunitz domain) may have mutations relative to a particular polypeptide described herein (e.g., a conservative or non-essential amino acid substitutions), which do not have a substantial effect on the polypeptide functions. Whether or not a particular substitution will be tolerated, i.e., will not adversely affect desired biological properties, such as binding activity can be determined as described in Bowie, et al. (1990) Science 247:1306-1310. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). It is possible for many framework and CDR amino acid residues to include one or more conservative substitutions.

A “non-essential” amino acid residue is a residue that can be altered from the wild-type sequence of the binding agent, e.g., the antibody, without abolishing or more preferably, without substantially altering a biological activity, whereas an “essential” amino acid residue results in such a change.

The terms “polypeptide” or “peptide” (which may be used interchangeably) refer to a polymer of three or more amino acids linked by a peptide bond, e.g., between 3 and 30, 12 and 60, or 30 and 300, or over 300 amino acids in length. The polypeptide may include one or more unnatural amino acids. Typically, the polypeptide includes only natural amino acids. A “protein” can include one or more polypeptide chains. Accordingly, the term “protein” encompasses polypeptides. A protein or polypeptide can also include one or more modifications, e.g., a glycosylation, amidation, phosphorylation, and so forth. The term “small peptide” can be used to describe a polypeptide that is between 3 and 30 amino acids in length, e.g., between 8 and 24 amino acids in length.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it.

The term “aryl” refers to an aromatic monocyclic, bicyclic, or tricyclic hydrocarbon ring system, wherein any ring atom capable of substitution can be substituted by a substituent. Examples of aryl moieties include, but are not limited to, phenyl, naphthyl, and anthracenyl.

The details of one or more embodiments of the invention are set forth in the accompanying drawings and the description below. Other features, objects, and advantages of the invention will be apparent from the description and drawings, and from the claims. All published patent applications, issued patents, and published references cited herein are incorporated by reference in their entirety. In particular, U.S. Pat. Nos. 5,663,143; 5,223,409, 6,010,080, and 6,333,402 are incorporated by reference in their entireties.

### BRIEF DESCRIPTION OF THE DRAWINGS

FIG. 1 is a set of graphs depicting plasma clearance curves for ¹²⁵I-labeled DX-890 and DX-890 PEGylated with different sized PEG, prepared at different pH and labeled with ¹²⁵I. Note the different time scales on the graphs for native DX-890 and PEGylated DX-890 conjugates.

FIG. 2 is a Log plot of plasma clearance curves for ¹²⁵I-labeled native and PEGylated DX-890 conjugates.

FIG. 3 is a set of SE-HPLC profiles using a Superose-12 column (Pharmacia) of plasma from animals injected with ¹²⁵I-DX-890. The insert within each panel shows time point, animal number and volume injected for HPLC analysis.

FIG. 4 is a set of SE-HPLC profiles using a Superose-12 column (Pharmacia) of plasma from animals injected with 20K PEGylated (pH 7.4) ¹²⁵I-DX-890. The insert within each panel shows time point, animal number and volume injected for HPLC analysis.

FIG. 5 is a set of SE-HPLC profiles using a Superose-12 column (Pharmacia) of plasma from animals injected with 30K PEGylated (pH 6) ¹²⁵I-DX-890. The insert within each panel shows time point, animal number and volume injected for HPLC analysis.

FIG. 6 is a set of SE-HPLC profiles using a Superose-12 column (Pharmacia) of plasma from animals injected with 20K PEGylated (pH 6) ¹²⁵I-DX-890. The insert within each panel shows time point, animal number and volume injected for HPLC analysis.

FIG. 7 is a set of graphs showing plasma clearance of ¹²⁵I lableled DX-890 and PEG-30-DX-890 in Rabbits. FIG. 7A shows shows results with % ID/mL plotted on a linear scale. FIG. 7B shows the same data with % ID/mL plotted on a log scale.

FIG. 8 is a set of HPLC profiles depicting results of SEC Analysis of ¹²⁵I-DX-890 in Rabbit Plasma Samples. The SE-HPLC profiles were generated using a Superose-12 column (Pharmacia) of plasma from animals injected with ¹²⁵I-DX-890. The insert within each panel shows time point and volume injected for HPLC analysis.

FIG. 9 depicts HPLC profiles from SEC Analysis of ¹²⁵I-PEG-30-DX-890 in Rabbit Plasma Samples. The SE-HPLC profiles were generated using a Superose-12 column Pharmacia) of plasma from animals injected with ¹²⁵I-PEG-30-DX-890. The insert within each panel shows time point and volume injected for HPLC analysis.

FIG. 10 presents linear extrapolations of the experimental data for mice (25 gm) and rabbits (2.5 Kg) to humans (70 Kg).

### DETAILED DESCRIPTION

The invention provides, in part, compounds that bind to and inhibit a protease (e.g., an elastase, e.g., a neutrophil elastase). The compounds include (i) a polypeptide that includes a Kunitz domain and (ii) a moiety (such as a polymer) that increases the molecular weight of the compounds relative to the polypeptide alone. The addition of the moiety to the compound can increase the in vivo circulating half life of the compound. In some embodiments, the compounds can inhibit neutrophil elastase with high affinity and selectivity.

Polymers

A variety of moieties can be used to increase the molecular weight of a polypeptide that includes a Kunitz domain or other protease inhibitor. In one embodiment, the moiety is a polymer, e.g., a water soluble and/or substantially non-antigenic polymer such as a homopolymer or a non-biological polymer. Substantially non-antigenic polymers include, e.g., polyalkylene oxides or polyethylene oxides. The moiety may improve stabilization and/or retention of the Kunitz domain in circulation, e.g., in blood, serum, lymph, or other tissues, e.g., by at least 1.5, 2, 5, 10, 50, 75, or 100 fold.

Suitable polymers can vary substantially by weight. For example, it is possible to use polymers having average molecular weights ranging from about 200 Daltons to about 40 kDa, e.g., 1-35 kDa or 10-32 kDa. In one embodiment, the average molecular weight of the polymer that is associated with the compound is between 15-25 kDa, or 18-22 kDa or about 20 kDa. In another embodiment, the average molecular weight of the polymer that is associated with the compound is 20-35 kDa, or 27-32 kDa, or about 30 kDa. The final molecular weight can also depend upon the desired effective size of the conjugate, the nature (e.g. structure, such as linear or branched) of the polymer, and the degree of derivatization.

A non-limiting list of exemplary polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. The polymer can be a hydrophilic polyvinyl polymers, e.g. polyvinylalcohol and polyvinylpyrrolidone. Additional useful polymers include polyoxyalkylenes such as polyoxyethylene, polyoxypropylene, and block copolymers of polyoxyethylene and polyoxypropylene (Pluronics); polylactic acid; polyglycolic acid; polymethacrylates; carbomers; branched or unbranched polysaccharides which comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, cellulose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparon. In some embodiments, the polymer includes a variety of different copolymer blocks.

The polypeptide that includes a Kunitz domain can be physically associated with the polymer in a variety of ways. Typically, the polypeptide is covalently linked to the polymer. For example, the polypeptide is conjugated to the polymer. Other compounds can also be attached to the same polymer, e.g., a cytotoxin, a label, or another targeting agent, e.g., another ligand that binds to the same target as the Kunitz domain or a ligand that binds to another target, e.g., a an unrelated ligand.

In one embodiment, the polymer is water soluble prior to conjugation to the polypeptide (although need not be). Generally, after conjugation to the polypeptide, the product is water soluble, e.g., exhibits a water solubility of at least about 0.01 mg/ml, and more preferably at least about 0.1 mg/ml, and still more preferably at least about 1 mg/ml. In addition, the polymer should not be highly immunogenic in the conjugate form, nor should it possess viscosity that is incompatible with intravenous infusion or injection if the conjugate is intended to be administered by such routes.

In one embodiment, the polymer contains only a single group which is reactive. This helps to avoid conjugation of one polymer to multiple protein molecules. Mono-activated, alkoxy-terminated polyalkylene oxides (PAO's), e.g., monomethoxy-terminated polyethylene glycols (mPEG's); C₁₋₄ alkyl-terminated polymers; and bis-activated polyethylene oxides (glycols) can be used for conjugation to the polypeptide. See, e.g., U.S. Pat. No. 5,951,974.

In its most common form, poly(ethylene glycol), PEG, is a linear or branched polyether terminated with hydroxyl groups. Linear PEG can have the following general structure:
HO—(CH₂CH₂O)ₙ—CH₂CH₂—OH
PEG can be synthesized by anionic ring opening polymerization of ethylene oxide initiated by nucleophilic attack of a hydroxide ion on the epoxide ring. Particularly useful for polypeptide modification is monomethoxy PEG, mPEG, having the general structure:
CH₃O—(CH₂CH₂O)ₙ—CH₂CH₂—OH

For further descriptions, see, e.g., Roberts et al. (2002) Advanced Drug Delivery Reviews 54:459-476. In one embodiment, the polymer units used for conjugation are mono-disperse or otherwise highly homogenous, e.g., present in a preparation in which 95% or all molecules are with 7, 5, 4, 3, 2, or 1 kDa of one another. In another embodiment, the polymer units are poly-disperse.

It is possible to select reaction conditions that reduce cross-linking between polymer units or conjugation to multiple polypeptides and to purify the reaction products through gel filtration or ion exchange chromatography to recover substantially homogenous derivatives, e.g., derivatives that include only a single Kunitz domain polypeptide. In other embodiments, the polymer contains two or more reactive groups for the purpose of linking multiple polypeptides (e.g., multiple units of the Kunitz domain polypeptide) to the polymer. Again, gel filtration or ion exchange chromatography can be used to recover the desired derivative in substantially homogeneous form.

In one embodiment, the polypeptide that includes a Kunitz domain is attached to a single molecule of PEG. For example, a Kunitz domain that inhibits elastase is attached to a single 30 kDa molecule of PEG.

A covalent bond can be used to attach a polypeptide (e.g., a polypeptide that includes a Kunitz domain) to a polymer, for example, conjugation to the N-terminal amino group. The polymer may be covalently bonded directly to the polypeptide without the use of a multifunctional (ordinarily bifunctional) crosslinking agent. Covalent binding to amino groups can be accomplished by known chemistries based upon cyanuric chloride, carbonyl diimidazole, aldehyde reactive groups (PEG alkoxide plus diethyl acetyl of bromoacetaldehyde; PEG plus DMSO and acetic anhydride, or PEG chloride plus the phenoxide of 4-hydroxybenzaldehyde, activated succinimidyl esters, activated dithiocarbonate PEG, 2,4,5-trichlorophenylcloroformate or P-nitrophenylcloroformate activated PEG.) Carboxyl groups can be derivatized by coupling PEG-amine using carbodiimide. Sulfhydryl groups can be derivatized by coupling to maleimido-substituted PEG (see, e.g., WO 97/10847) or PEG-maleimide (e.g., commercially available from Shearwater Polymers, Inc., Huntsville, Ala.).

Functionalized PEG polymers that can be attached to a polypeptide that includes Kunitz domain include polymers that are commercially available, e.g., from Shearwater Polymers, Inc. (Huntsville, Ala.). Such PEG derivatives include, e.g., amino-PEG, PEG amino acid esters, PEG-hydrazide, PEG-thiol, PEG-succinate, carboxymethylated PEG, PEG-propionic acid, PEG amino acids, PEG succinimidyl succinate, PEG succinimidyl propionate, succinimidyl ester of carboxymethylated PEG, succinimidyl carbonate of PEG, succinimidyl esters of amino acid PEGs, PEG-oxycarbonylimidazole, PEG-nitrophenyl carbonate, PEG tresylate, PEG-glycidyl ether, PEG-aldehyde, PEG vinylsulfone, PEG-maleimide, PEG-orthopyridyl-disulfide, heterofunctional PEGs, PEG vinyl derivatives, PEG silanes, and PEG phospholides. The reaction conditions for coupling these PEG derivatives may vary depending on the polypeptide, the desired degree of PEGylation, and the PEG derivative utilized. Some factors involved in the choice of PEG derivatives include: the desired point of attachment, hydrolytic stability and reactivity of the derivatives, stability, toxicity and antigenicity of the linkage, suitability for analysis, etc.

The conjugates of a polypeptide that includes a Kunitz domain and a polymer can be separated from the unreacted starting materials using chromatographic methods, e.g., by gel filtration or ion exchange chromatography, e.g., HPLC. Heterologous species of the conjugates are purified from one another in the same fashion. Resolution of different species is also possible due to the difference in the ionic properties of the unreacted amino acids. See, e.g., WO 96/34015.

Kunitz Domains

As used herein, a “Kunitz domain” is a polypeptide domain having at least 51 amino acids and containing at least two, and preferably three, disulfides. The domain is folded such that the first and sixth cysteines, the second and fourth, and the third and fifth cysteines form disulfide bonds (e.g., in a Kunitz domain having 58 amino acids, cysteines can be present at positions corresponding to amino acids 5, 14, 30, 38, 51, and 55, according to the number of the BPTI sequence provided below, and disulfides can form between the cysteines at position 5 and 55, 14 and 38, and 30 and 51), or, if two disulfides are present, they can form between a corresponding subset of cysteines thereof. The spacing between respective cysteines can be within 7, 5, 4, 3 or 2 amino acids of the following spacing between positions corresponding to: 5 to 55, 14 to 38, and 30 to 51, according to the numbering of the BPTI sequence provided below. The BPTI sequence can be used a reference to refer to specific positions in any generic Kunitz domain. Comparison of a Kunitz domain of interest to BPTI can be performed by identifying the best fit alignment in which the number of aligned cysteines in maximized.

The 3D structure (at high resolution) of the Kunitz domain of BPTI is known. One of the X-ray structures is deposited in the Brookhaven Protein Data Bank as “6PTI”. The 3D structure of some BPTI homologues (Eigenbrot et al., (1990) Protein Engineering, 3(7):591-598; Hynes et al., (1990) Biochemistry, 29:10018-10022) are known. At least seventy Kunitz domain sequences are known. Known human homologues include three Kunitz domains of LACI (Wun et al., (1988) J. Biol. Chem.; 263(13):6001-6004; Girard et al., (1989) Nature, 338:518-20; Novotny et al, (1989) J. Biol. Chem., 264(31):18832-18837) two Kunitz domains of Inter-α-Trypsin Inhibitor, APP-I (Kido et al., (1988) J. Biol. Chem., 263(34):18104-18107), a Kunitz domain from collagen, and three Kunitz domains of TFPI-2 (Sprecher et al., (1994) PNAS USA, 91:3353-3357). LACI is a human serum phosphoglycoprotein with a molecular weight of 39 kDa (amino acid sequence in Table 1) containing three Kunitz domains.

TABLE 1
Exemplary Natural Kunitz Domains

| LACI: | | |
|---|---|---|
| (SEQ ID NO. 1) | 1 | MIYTMKKVHA LWASVCLLLN LAPAPLNAds eedeehtiit dtelpplklM |
| | 51 | HSFCAFKADD GPCKAIMKRF FFNIFTRQCE EFIYGGCEGN QNRFESLEEC |
| | 101 | KKMCTRDnan riikttlqqe kpdfCfleed pgiCrgyitr yfynnqtkgC |
| | 151 | erfkyggClg nmnnfetlee CkniCedgpn gfqvdnygtq lnavnnsltp |
| | 201 | qstkvpslfe fhgpswCltp adrglCrane nrfyynsvig kCrpfkysgC |
| | 251 | ggnennftsk qeClraCkkg fiqriskggl iktkrkrkkq rvkiayeeif |
| | 301 | vknm |

The signal sequence (1-28) is uppercase and underscored
LACI-K1 is uppercase
LACI-K2 is underscored
LACI-K3 is bold

| | |
|---|---|
| BPTI | 1 2 3 4 5 |
| (SEQ ID NO:2) | 1234567890123456789012345678901234567890123456789012345678 |
| | RPDFCLEPPYTGPCKARIIRYFYNAKAGLCQTFVYGGCRAKRNNFKSAEDCMRTCGGA |

The Kunitz domains above are referred to as LACI-K1 (residues 50 to 107), LACI-K2 (residues 121 to 178), and LACI-K3 (213 to 270). The cDNA sequence of LACI is reported in Wun et al. (J. Biol. Chem., 1988, 263(13):6001-6004). Girard et al. (Nature, 1989, 338:518-20) reports mutational studies in which the P1 residues of each of the three Kunitz domains were altered. LACI-K1 inhibits Factor VIIa (F.VIIa) when F.VIIa is complexed to tissue factor and LACI-K2 inhibits Factor Xa.

A variety of methods can be used to identify a Kunitz domain from a sequence database. For example, a known amino acid sequence of a Kunitz domain, a consensus sequence, or a motif (e.g., the ProSite Motif) can be searched against the GenBank sequence databases (National Center for Biotechnology Information, National Institutes of Health, Bethesda Md.), e.g., using BLAST; against Pfam database of HMMs (Hidden Markov Models) (e.g., using default parameters for Pfam searching; against the SMART database; or against the ProDom database. For example, the Pfam Accession Number PF00014 of Pfam Release 9 provides numerous Kunitz domains and an HMM for identify Kunitz domains. A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314. The SMART database (Simple Modular Architecture Research Tool, EMBL, Heidelberg, Del.) of HMMs as described in Schultz et al. (1998), Proc. Natl. Acad. Sci. USA 95:5857 and Schultz et al. (2000) Nucl. Acids Res 28:231. The SMART database contains domains identified by profiling with the hidden Markov models of the HMMer2 search program (R. Durbin et al. (1998) Biological sequence analysis: probabilistic models of proteins and nucleic acids. Cambridge University Press). The database also is annotated and monitored. The ProDom protein domain database consists of an automatic compilation of homologous domains (Corpet et al. (1999), Nucl. Acids Res. 27:263-267). Current versions of ProDom are built using recursive PSI-BLAST searches (Altschul et al. (1997) Nucleic Acids Res. 25:3389-3402; Gouzy et al. (1999) Computers and Chemistry 23:333-340.) of the SWISS-PROT 38 and TREMBL protein databases. The database automatically generates a consensus sequence for each domain. Prosite lists the Kunitz domain as a motif and identifies proteins that include a Kunitz domain. See, e.g., Falquet et al. Nucleic Acids Res. 30:235-238(2002).

Kunitz domains interact with target protease using, primarily, amino acids in two loop regions. The first loop region is between about residues corresponding to amino acids 15-20 of BPTI. The second loop region is between about residues corresponding to amino acids 31 to 37 of BPTI. An exemplary library of Kunitz domains varies one or more amino acid positions in the first and/or second loop regions. Particularly useful positions to vary include: positions 13, 16, 17, 18, 19, 31, 32, 34, and 39 with respect to the sequence of BPTI. At least some of these positions are expected to be in close contact with the target protease

Conversely, residues that are not at these particular positions or which are not in the loop regions may tolerate a wider range of amino acid substitution (e.g., conservative and/or non-conservative substitutions) than these amino acid positions.

Elastase-Inhibiting Kunitz Domains

One exemplary polypeptide that binds to an inhibits human neutrophil elastase (hNE) is DX-890 (also known as “EPI-hNE4”). DX-890 is a highly specific and potent (Ki=4×10⁻¹² M) inhibitor of human neutrophil elastase (hNE). DX-890 includes the following amino acid sequence:

| | |
|---|---|
| Glu Ala Cys Asn Leu Pro Ile Val Arg | (SEQ ID NO:1) |
| Gly Pro Cys Ile Ala Phe Phe Pro Arg | |
| Trp Ala Phe Asp Ala Val Lys Gly Lys | |
| Cys Val Leu Phe Pro Tyr Gly Gly Cys | |
| Gln Gly Asn Gly Asn Lys Phe Tyr Ser | |
| Glu Lys Glu Cys Arg Glu Tyr Cys Gly | |
| Val Pro | |

DX-890 is derived from the second Kunitz-type domain of inter-α-inhibitor protein (ITI-D2) and can be produced by fermentation in Pichia pastoris. It includes 56 amino acids, with a predicted MW of 6,237 Daltons. DX-890 is resistant to oxidative and proteolytic inactivation.

There are also known correlations between the structure of DX-890 and its ability to bind to hNE. See, e.g., U.S. Pat. No. 5,663,143. U.S. Pat. No. 5,663,143 also describes other Kunitz domains that inhibit elastase. These and related domains (e.g., domains at least 70, 75, 80, 85, 90, or 95% identical) can also be used.

TABLE 2
Exemplary Amino Acids for hNE inhibitors
Some preferred Amino acids in hNE-inhibiting Kunitz domains Position
Allowed amino acids at amino acid positions corresponding to
respective positions in BPTI

| | |
|---|---|
| 5 | C |
| 10 | YSVN |
| 11 | TARQP |
| 12 | G |
| 13 | PAV |
| 14 | C |
| 15 | IV |
| 16 | AG |
| 17 | FILVM |
| 18 | F |
| 19 | PSQKR |
| 20 | R |
| 21 | YWF |
| 30 | C |
| 31 | QEV |
| 32 | TLP |
| 33 | F |
| 34 | VQP |
| 35 | Y |
| 36 | G |
| 37 | G |
| 38 | C |
| 39 | MQ |
| 40 | GA |
| 41 | N |
| 42 | G |
| 43 | N |
| 45 | F |
| 51 | C |
| 55 | C |

Identifying Kunitz Domains and Other Protease Inhibitors

A variety of methods can be used to identify a protein that binds to and/or inhibits a protease. These methods can be used to identify natural and non-naturally occurring Kunitz domains that can be used as components of the compounds described herein.

For example, a Kunitz domain can be identified from a library of proteins in which each of a plurality of library members includes a varied Kunitz domain. A variety of amino acids can be varied in the domain. See, e.g., U.S. Pat. No. 5,223,409; U.S. Pat. No. 5,663,143, and U.S. Pat. No. 6,333,402. Kunitz domains can varied, e.g., using DNA mutagenesis, DNA shuffling, chemical synthesis of oligonucleotides (e.g., using codons as subunits), and cloning of natural genes. See, e.g., U.S. Pat. No. 5,223,409 and U.S. 2003-0129659.

The library can be an expression library that is used to produce proteins. The proteins can be arrayed, e.g., using a protein array. U.S. Pat. No. 5,143,854; De Wildt et al. (2000) Nat. Biotechnol. 18:989-994; Lueking et al. (1999) Anal. Biochem. 270:103-111; Ge (2000) Nucleic Acids Res. 28, e3, I-VII; MacBeath and Schreiber (2000) Science 289:1760-1763; WO 0/98534, WO01/83827, WO02/12893, WO 00/63701, WO 01/40803 and WO 99/51773.

The proteins can also be displayed on a replicable genetic package, e.g., in the form of a phage library such as a phage display, yeast display library, ribosome display, or nucleic acid-protein fusion library. See, e.g., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem 274:18218-30; Hoogenboom et al. (1998) Immunotechnology 4:1-20; Hoogenboom et al. (2000) Immunol Today 2:371-8; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Rebar et al. (1996) Methods Enzymol. 267:129-49; Hoogenboom et al. (1991) Nuc Acid Res 19:4133-4137; and Barbas et al. (1991) PNAS 88:7978-7982 for examples of phage display and other methods. See, e.g., Boder and Wittrup (1997) Nat. Biotechnol. 15:553-557 and WO 03/029456 for examples of yeast cell display and other methods. See, e.g., Mattheakis et al. (1994) Proc. Natl. Acad. Sci. USA 91:9022 and Hanes et al. (2000) Nat Biotechnol. 18:1287-92; Hanes et al. (2000) Methods Enzymol. 328:404-30. and Schaffitzel et al. (1999) J Immunol Methods. 231(1-2):119-35 for examples of ribosome display and other methods. See, e.g., Roberts and Szostak (1997) Proc. Natl. Acad. Sci. USA 94:12297-12302, and U.S. Pat. No. 6,207,446 for examples of nucleic acid-protein fusions. Such libraries can be screened in a high throughput format. See, e.g., U.S. 2003-0129659.

Screening Display Libraries

This section describes exemplary methods of screening a display library to identify a polypeptide that interacts with an elastase. These methods can be modified to identify other polypeptides that interact with other targets, e.g., other proteases or other proteins. The methods can also be modified and used in combination with other types of libraries, e.g., an expression library or a protein array, and so forth.

In an exemplary display library screen, a phage library is contacted with and allowed to bind to the target elastase protein (e.g., an active or an inactivated form (e.g., mutant or chemically inactivated protein) or a fragment thereof). To facilitate separation of binders and non-binders in the screening process, it is often convenient to immobilize the elastase on a solid support, although it is also possible to first permit binding to elastase in solution and then segregate binders from non-binders by coupling the target compound to a support. By way of illustration, when incubated in the presence of the elastase, phage displaying a polypeptide that interacts with elastase form a complex with the elastase immobilized on a solid support whereas non-binding phage remain in solution and may be washed away with buffer. Bound phage may then be liberated from the elastase by a number of means, such as changing the buffer to a relatively high acidic or basic pH (e.g., pH 2 or pH 10), changing the ionic strength of the buffer, adding denaturants, adding a competitor, adding a host cell which can be infected, or other known means.

For example, to identify elastase-binding peptides, elastase can be adsorbed to a solid surface, such as the plastic surface of wells in a multi-well assay plate. Subsequently, an aliquot of a phage display library is added to a well under appropriate conditions that maintain the structure of the immobilized elastase and the phage, such as pH 6-7. Phage in the libraries that display polypeptides that bind the immobilized elastase are bound to the elastase and are retained in the well. Non-binding phage can be removed. It is also possible to include a blocking agent or competing ligand during the binding of the phage library to the immobilized elastase.

Phage bound to the immobilized elastase may then be eluted by washing with a buffer solution having a relatively strong acid pH (e.g., pH 2) or an alkaline pH (e.g., pH 8-9). The solutions of recovered phage that are eluted from the elastase are then neutralized and may, if desired, be pooled as an enriched mixed library population of phage displaying elastase binding peptides. Alternatively the eluted phage from each library may be kept separate as a library-specific enriched population of elastase binders. Enriched populations of phage displaying elastase binding peptides may then be grown up by standard methods for further rounds of screening and/or for analysis of peptide displayed on the phage and/or for sequencing the DNA encoding the displayed binding peptide.

One of many possible alternative screening protocols uses elastase target molecules that are biotinylated and that can be captured by binding to streptavidin, for example, coated on particles.

Recovered phage may then be amplified by infection of bacterial cells, and the screening process may be repeated with the new pool of phage that is now depleted in non-elastase binders and enriched in elastase binders. The recovery of even a few binding phage may be sufficient to carry the process to completion. After a few rounds of selection, the gene sequences encoding the binding moieties derived from selected phage clones in the binding pool are determined by conventional methods, revealing the peptide sequence that imparts binding affinity of the phage to the target. An increase in the number of phage recovered after each round of selection and the recovery of closely related sequences indicate that the screening is converging on sequences of the library having a desired characteristic.

After a set of binding polypeptides is identified, the sequence information may be used to design other, secondary libraries. For example, the secondary libraries can explore a smaller segment of sequence space in more detail than the initial library. In some embodiments the a secondary library includes proteins that are biased for members having additional desired properties, e.g., sequences that have a high percentage identity to a human protein.

Display technology can also be used to obtain polypeptides that are specific to particular epitopes of a target. This can be done, for example, by using competing non-target molecules that lack the particular epitope or are mutated within the epitope, e.g., with alanine. Such non-target molecules can be used in a negative selection procedure as described below, as competing molecules when binding a display library to the target, or as a pre-elution agent, e.g., to capture in a wash solution dissociating display library

Iterative Selection. In one preferred embodiment, display library technology is used in an iterative mode. A first display library is used to identify one or more proteins that interacts with a target. These identified proteins are then varied using a mutagenesis method to form a second display library. Higher affinity proteins are then selected from the second library, e.g., by using higher stringency or more competitive binding and washing conditions.

In some implementations, the mutagenesis is targeted to regions known or likely to be at the binding interface. Some exemplary mutagenesis techniques include: error-prone PCR (Leung et al. (1989) Technique 1:11-15), recombination, DNA shuffling using random cleavage (Stemmer (1994) Nature 389-391; termed “nucleic acid shuffling”), RACHITT™ (Coco et al. (2001) Nature Biotech. 19:354), site-directed mutagenesis (Zoller et al. (1987) Nucl Acids Res 10:6487-6504), cassette mutagenesis (Reidhaar-Olson (1991) Methods Enzymol. 208:564-586) and incorporation of degenerate oligonucleotides (Griffiths et al. (1994) EMBO J. 13:3245). For Kunitz domains, many positions near the binding interface are known. Such positions include, for example, positions 13, 16, 17, 18, 19, 31, 32, 34, and 39 with respect to the sequence of BPTI. (according to the BPTI numbering in U.S. Pat. No. 6,333,402). Such positions can be held constant and other positions can be varied or these positions themselves may be varied.

In one example of iterative selection, the methods described herein are used to first identify a proteins from a display library that binds a elastase with at least a minimal binding specificity for a target or a minimal activity, e.g., an equilibrium dissociation constant for binding of greater than 1 nM, 10 nM, or 100 nM. The nucleic acid sequences encoding the initial identified proteins are used as a template nucleic acid for the introduction of variations, e.g., to identify a second protein ligand that has enhanced properties (e.g., binding affinity, kinetics, or stability) relative to the initial protein ligand.

Off-Rate Selection. Since a slow dissociation rate can be predictive of high affinity, particularly with respect to interactions between proteins and their targets, the methods described herein can be used to isolate proteins with a desired kinetic dissociation rate (i.e. reduced) for a binding interaction to a target.

To select for slow dissociating proteins from a display library, the library is contacted to an immobilized target, e.g., immobilized elastase. The immobilized target is then washed with a first solution that removes non-specifically or weakly bound biomolecules. Then the immobilized target is eluted with a second solution that includes a saturation amount of free target, i.e., replicates of the target that are not attached to the particle. The free target binds to biomolecules that dissociate from the target. Rebinding is effectively prevented by the saturating amount of free target relative to the much lower concentration of immobilized target.

The second solution can have solution conditions that are substantially physiological or that are stringent. Typically, the solution conditions of the second solution are identical to the solution conditions of the first solution. Fractions of the second solution are collected in temporal order to distinguish early from late fractions. Later fractions include biomolecules that dissociate at a slower rate from the target than biomolecules in the early fractions.

Further, it is also possible to recover display library members that remain bound to the target even after extended incubation. These can either be dissociated using chaotropic conditions or can be amplified while attached to the target. For example, phage bound to the target can be contacted to bacterial cells.

Selecting or Screening for Specificity. The display library screening methods described herein can include a selection or screening process that discards display library members that bind to a non-target molecule, e.g., a protease other than elastase, such as trypsin. In one embodiment, the non-target molecule is elastase that has been activated by treatment with an irreversibly bound inhibitor, e.g., a covalent inhibitor.

In one implementation, a so-called “negative selection” step or “depletion” is used to discriminate between the target and related non-target molecule and a related, but distinct non-target molecules. The display library or a pool thereof is contacted to the non-target molecule. Members of the sample that do not bind the non-target are collected and used in subsequent selections for binding to the target molecule or even for subsequent negative selections. The negative selection step can be prior to or after selecting library members that bind to the target molecule.

In another implementation, a screening step is used. After display library members are isolated for binding to the target molecule, each isolated library member is tested for its ability to bind to a non-target molecule (e.g., a non-target listed above). For example, a high-throughput ELISA screen can be used to obtain this data. The ELISA screen can also be used to obtain quantitative data for binding of each library member to the target. The non-target and target binding data are compared (e.g., using a computer and software) to identify library members that specifically bind to the target.

Modifying and Varying Polypeptides

It is also possible to vary a protein that interacts with elastase to obtain useful variant protein that interact with elastase. Typically, a number of variants are possible. A variant can be prepared and then tested, e.g., using a binding assay described herein (such as fluorescence anisotropy).

One type of variant is a truncation of a ligand described herein or isolated by a method described herein. In this example, the variant is prepared by removing one or more amino acid residues of the ligand from the N or C terminus. In some cases, a series of such variants is prepared and tested. Information from testing the series is used to determine a region of the ligand that is essential for binding the elastase protein. A series of internal deletions or insertions can be similarly constructed and tested. For Kunitz domains, it can be possible to remove, e.g., between one and five residues or one and three residues that are N-terminal to C₅, the first cysteine, and between one and five residues or one and three residues that are C-terminal to C₅₅, the final cysteine, wherein each of the cysteines corresponds to a respectively numbered cysteine in BPTI.

Another type of variant is a substitution. In one example, the ligand is subjected to alanine scanning to identify residues that contribute to binding activity. In another example, a library of substitutions at one or more positions is constructed. The library may be unbiased or, particularly if multiple positions are varied, biased towards an original residue. In some cases, the substitutions are all conservative substitutions.

Another type of variant includes one or more non-naturally occurring amino acids. Such variant ligands can be produced by chemical synthesis or modification. One or more positions can be substituted with a non-naturally occurring amino acid. In some cases, the substituted amino acid may be chemically related to the original naturally occurring residue (e.g., aliphatic, charged, basic, acidic, aromatic, hydrophilic) or an isostere of the original residue.

It may also be possible to include non-peptide linkages and other chemical modifications. For example, part or all of the ligand may be synthesized as a peptidomimetic, e.g., a peptoid (see, e.g., Simon et al. (1992) Proc. Natl. Acad. Sci. USA 89:9367-71 and Horwell (1995) Trends Biotechnol. 13:132-4). See also other modifications discussed below.

Characterization of Binding Interactions

The binding properties of a protein (e.g., a polypeptide that includes a Kunitz domain) can be readily assessed using various assay formats. For example, the binding property of a protein can be measured in solution by fluorescence anisotropy, which provides a convenient and accurate method of determining a dissociation constant (K_{D}) or association constant (Ka) of the protein for a particular target. In one such procedure, the protein to be evaluated is labeled with fluorescein. The fluorescein-labeled protein is mixed in wells of a multi-well assay plate with various concentrations of the particular target (e.g., elastase). Fluorescence anisotropy measurements are carried out using a fluorescence polarization plate reader.

ELISA. The binding interactions can also be analyzed using an ELISA assay. For example, the protein to be evaluated is contacted to a microtitre plate whose bottom surface has been coated with the target, e.g., a limiting amount of the target. The molecule is contacted to the plate. The plate is washed with buffer to remove non-specifically bound molecules. Then the amount of the protein bound to the plate is determined by probing the plate with an antibody that recognizes the protein. For example, the protein can include an epitope tag. The antibody can be linked to an enzyme such as alkaline phosphatase, which produces a colorimetric product when appropriate substrates are provided. In the case where a display library member includes the protein to be tested, the antibody can recognize a region that is constant among all display library members, e.g., for a phage display library member, a major phage coat protein.

Homogeneous Assays. A binding interaction between a protein and a particular target can be analyzed using a homogenous assay, i.e., after all components of the assay are added, additional fluid manipulations are not required. For example, fluorescence energy transfer (FET) can be used as a homogenous assay (see, for example, Lakowicz et al., U.S. Pat. No. 5,631,169; Stavrianopoulos, et al., U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter). By titrating the amount of the first or second binding molecule, a binding curve can be generated to estimate the equilibrium binding constant.

Surface Plasmon Resonance (SPR). A binding interaction between a protein and a particular target can be analyzed using SPR. For example, after sequencing of a display library member present in a sample, and optionally verified, e.g., by ELISA, the displayed protein can be produced in quantity and assayed for binding the target using SPR. SPR or real-time Biomolecular Interaction Analysis (BIA) detects biospecific interactions in real time, without labeling any of the interactants (e.g., BIAcore). Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander, S. and Urbaniczky, C. (1991) Anal. Chem. 63:2338-2345; Szabo et al. (1995) Curr. Opin. Struct. Biol. 5:699-705.

Information from SPR can be used to provide an accurate and quantitative measure of the equilibrium dissociation constant (K_{d}), and kinetic parameters, including kₒₙ and k_{off}, for the binding of a biomolecule to a target. Such data can be used to compare different biomolecules. For example, proteins selected from a display library can be compared to identify individuals that have high affinity for the target or that have a slow k_{off}. This information can also be used to develop structure-activity relationship (SAR) if the biomolecules are related. For example, if the proteins are all mutated variants of a single parental antibody or a set of known parental antibodies, variant amino acids at given positions can be identified that correlate with particular binding parameters, e.g., high affinity and slow k_{off}.

Additional methods for measuring binding affinities include fluorescence polarization (FP) (see, e.g., U.S. Pat. No. 5,800,989), nuclear magnetic resonance (NMR), and binding titrations (e.g., using fluorescence energy transfer).

Other solution measures for studying binding properties include fluorescence resonance energy transfer (FRET) and NMR.

Characterization of Elastase Inhibition

With respect to embodiments in which the compound includes a polypeptide that has a Kunitz domain specific for elastase, it may be useful to characterize the ability of the polypeptide to inhibit elastase.

Kunitz domains can be screened for binding to elastase and for inhibition of elastase proteolytic activity. Kunitz domains can be selected for their potency and selectivity of inhibition of elastase. In one example, elastase and its substrate are combined under assay conditions permitting reaction of the protease with its substrate. The assay is performed in the absence of the Kunitz domain, and in the presence of increasing concentrations of the Kunitz domain. The concentration of test compound at which 50% of the elastase activity is inhibited by the test compound is the IC₅₀ value (Inhibitory Concentration) or EC₅₀ (Effective Concentration) value for that compound. Within a series or group of Kunitz domain, those having lower IC₅₀ or EC₅₀ values are considered more potent inhibitors of the elastase than those compounds having higher IC₅₀ or EC₅₀ values. Preferred compounds according to this aspect have an IC₅₀ value of 100 nM or less as measured in an in vitro assay for inhibition of elastase activity.

Kunitz domain can also be evaluated for selectivity toward elastase. A test compound is assayed for its potency toward a panel of serine proteases and other enzymes and an IC₅₀ value is determined for each peptide. A Kunitz domain that demonstrates a low IC₅₀ value for the elastase enzyme, and a higher IC₅₀ value for other enzymes within the test panel (e.g., trypsin, plasmin, kallikrein), is considered to be selective toward elastase. Generally, a compound is deemed selective if its IC₅₀ value is at least one order of magnitude less than the next smallest IC₅₀ value measured in the panel of enzymes.

Specific methods for evaluating inhibition of elastase are described in Example 1.

It is also possible to evaluate Kunitz domain activity in vivo or in samples of subjects to which a compound described herein has been administered.

Protease Targets

Human neutrophil elastase consists of approximately 218 amino acid residues, contains 2 asparagine-linked carbohydrate side chains, and is joined together by 2 disulfide bonds (Sinha, S., et al. Proc. Nat. Acad. Sci. 84: 2228-2232, 1987). It is normally synthesized in the developing neutrophil as a proenzyme but stored in the primary granules in its active form, ready with full enzymatic activity when released from the granules, normally at sites of inflammation (Gullberg U, et al. Eur J Haematol. 1997; 58:137-153; Borregaard N, Cowland J B. Blood. 1997; 89:3503-3521).

Synthetic Peptides

The binding ligand can include an artificial peptide of 32 amino acids or less that independently binds to a target molecule, e.g., a target protease, e.g., elastase. Some synthetic peptides can include one or more disulfide bonds. Other synthetic peptides, so-called “linear peptides,” are devoid of cysteines. Synthetic peptides may have little or no structure in solution (e.g., unstructured), heterogeneous structures (e.g., alternative conformations or “loosely structured), or a singular native structure (e.g., cooperatively folded). Some synthetic peptides adopt a particular structure when bound to a target molecule. Some exemplary synthetic peptides are so-called “cyclic peptides” that have at least a disulfide bond and, for example, a loop of about 4 to 12 non-cysteine residues. Exemplary peptides are less than 28, 24, 20, or 18 amino acids in length.

Peptide sequences that independently bind a molecular target, e.g., a protease such as elastase, can be selected from a display library or an array of peptides. See, e.g., U.S. 2003-0129659. After identification, such peptides can be produced synthetically or by recombinant means. The sequences can be incorporated (e.g., inserted, appended, or attached) into longer sequences. The sequences can be tested for ability to inhibit a target, e.g., a protease.

Peptide ligands that bind to a protease can include six or more amino acids. The amino acids subunits can be naturally occurring (e.g., one of the twenty commonly used naturally occurring amino acids) or non-naturally occurring) amino acids, or combinations thereof. Similarly, the amino acid sequence can be naturally occurring or not naturally occurring. Peptide analogs can also be used as non-peptide elastase ligands with properties analogous to those of the template peptide. See, e.g., Luthman et al., A Textbook of Drug Design and Development, 14: 386-406, 2nd Ed., Harwood Academic Publishers (1996); Joachim Grante (1994) Angew. Chem. Int Ed. Engl., 33: 1699-1720; Fauchere (1986) J. Adv. Drug Res., 15:29; Veber and Freidinger (1985) TINS, p. 392; and Evans et al. (1987) J. Med. Chem. 30:1229); Roberts et al. (1983) Unusual Amino Acids in Peptide Synthesis, 5 (6):341-449; Morgan et al. (1989) Ann. Rep. Med. Chem., 24:243-252; Murray et al. (1995) Burger's Medicinal Chemistry and Drug Discovery, 5th ed., VoL 1, Manfred E. Wolf, ed., John Wiley and Sons, Inc.; Zallipsky (1995) Bioconjugate Chem., 6:150-165; Monfardini et al. (1995) Bioconjugate Chem., 6:62-69; U.S. Pat. Nos. 4,640,835; 4,496,689; 4,301,144; 4,670,417; 4,791,192; 4,179,337 and WO 95/34326; Hruby et al. (1990), Biochem J., 268 (2): 249-262.

Other Exemplary Scaffolds

Other exemplary scaffolds that can be variegated to produce a protein that binds to elastase can include: extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases particularly inactivated proteases), RNase; chaperones, e.g., thioredoxin, and heat shock proteins; and intracellular signaling domains (such as SH2 and SH3 domains) and antibodies (e.g., Fab fragments, single chain Fv molecules (scFV), single domain antibodies, camelid antibodies, and camelized antibodies); T-cell receptors and MHC proteins.

U.S. Pat. No. 5,223,409 also describes a number of so-called “mini-proteins,” e.g., mini-proteins modeled after α-conotoxins (including variants GI, GII, and MI), mu-(GIIIA, GIIIB, GIIIC) or OMEGA-(GVIA, GVIB, GVIC, GVIIA, GVIIB, MVIIA, MVIIB, etc.) conotoxins.

Protein Production

Recombinant production of polypeptides. Standard recombinant nucleic acid methods can be used to express a polypeptide component of a compound described herein (e.g., a polypeptide that includes a Kunitz domain). Generally, a nucleic acid sequence encoding the polypeptide is cloned into a nucleic acid expression vector. If the polypeptide is sufficiently small, e.g., the protein is a peptide of less than 50 amino acids, the protein can be synthesized using automated organic synthetic methods.

The expression vector for expressing the polypeptide can include a segment encoding the polypeptide and regulatory sequences, for example, a promoter, operably linked to the coding segment. Suitable vectors and promoters are known to those of skill in the art and are commercially available for generating the recombinant constructs of the present invention. See, for example, the techniques described in Sambrook & Russell, Molecular Cloning: A Laboratory Manual, 3^{rd} Edition, Cold Spring Harbor Laboratory, N.Y. (2001) and Ausubel et al., Current Protocols in Molecular Biology (Greene Publishing Associates and Wiley Interscience, N.Y. (1989).

Scopes (1994) Protein Purification: Principles and Practice, New York:Springer-Verlag and other texts provide a number of general methods for purifying recombinant (and non-recombinant) proteins.

Synthetic production of peptides. The polypeptide component of a compound can also be produced by synthetic means. See, e.g., Merrifield (1963) J. Am. Chem. Soc., 85: 2149. For example, the molecular weight of synthetic peptides or peptide mimetics can be from about 250 to about 8,0000 Daltons. A peptide can be modified, e.g., by attachment to a moiety that increases the effective molecular weight of the peptide. If the peptide is oligomerized, dimerized and/or derivatized, e.g., with a hydrophilic polymer (e.g., to increase the affinity and/or activity of the peptides), its molecular weights can be greater and can range anywhere from about 500 to about 50,000 Daltons.

Pharmaceutical Compositions

Also featured is a composition, e.g., a pharmaceutically acceptable composition, that includes a compound that contains (i) a polypeptide that includes a Kunitz domain and (ii) a moiety (such as a polymer) that increases the molecular weight of the compound. In one embodiment, the polypeptide binds to a protease such as elastase. As used herein, “pharmaceutical compositions” encompass compounds (e.g., labeled compounds) for diagnostic (e.g., in vivo imaging) use as well as compounds for therapeutic or prophylactic use.

As used herein, “pharmaceutically acceptable carrier” includes any and all solvents, dispersion media, coatings, antibacterial and antifungal agents, isotonic and absorption delaying agents, and the like that are physiologically compatible. In one embodiment, the carrier is other than water. Preferably, the carrier is suitable for intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for administration of humans with antibodies. The preferred mode of administration is parenteral (e.g., intravenous, subcutaneous, intraperitoneal, intramuscular). In a preferred embodiment, the compound is administered by intravenous infusion or injection. In another preferred embodiment, the compound is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Pharmaceutical compositions typically must be sterile and stable under the conditions of manufacture and storage. A pharmaceutical composition can also be tested to insure it meets regulatory and industry standards for administration. For example, endotoxin levels in the preparation can be tested using the Limulus amebocyte lysate assay (e.g., using the kit from Bio Whittaker lot # 7L3790, sensitivity 0.125 EU/mL) according to the USP 24/NF 19 methods. Sterility of pharmaceutical compositions can be determined using thioglycollate medium according to the USP 24/NF 19 methods. For example, the preparation is used to inoculate the thioglycollate medium and incubated at 35° C. for 14 or more days. The medium is inspected periodically to detect growth of a microorganism.

The composition can be formulated as a solution, microemulsion, dispersion, liposome, or other ordered structure suitable to high drug concentration. Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The compounds described herein can be administered by a variety of methods known in the art. For many applications, the route/mode of administration is intravenous injection or infusion. For example, for therapeutic applications, the compound can be administered by intravenous infusion at a rate of less than 30, 20, 10, 5, or 1 mg/min to reach a dose of about 1 to 100 mg/m² or 7 to 25 mg/m². The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978. Pharmaceutical formulation is a well-established art, and is further described in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

In certain embodiments, the composition may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compound may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

Pharmaceutical compositions can be administered with medical devices known in the art. For example, in a preferred embodiment, a pharmaceutical composition of the invention can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules are also known.

In certain embodiments, the compound can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties that are selectively transported into specific cells or organs, thus enhance targeted drug delivery (see, e.g., V. V. Ranade (1989) J. Clin. Pharmacol. 29:685).

Also within the scope of the invention are kits comprising a composition described herein (e.g., a composition a compound that contains (i) a polypeptide that includes a Kunitz domain and (ii) a moiety (such as a polymer) that increases the molecular weight of the compound) and instructions for use, e.g., treatment, prophylactic, or diagnostic use. In one embodiment, the kit includes (a) the compound, e.g., a composition that includes the compound, and, optionally, (b) informational material. The informational material can be descriptive, instructional, marketing or other material that relates to the methods described herein and/or the use of the compound for the methods described herein. For example, the informational material describes methods for administering the compound to reduce elastase activity or to treat or prevent a pulmonary disorder (e.g., CF or COPD), an inflammatory disorder (e.g., IBD), or a disorder characterized by excessive elastase activity.

In one embodiment, the informational material can include instructions to administer the compound in a suitable manner, e.g., in a suitable dose, dosage form, or mode of administration (e.g., a dose, dosage form, or mode of administration described herein). In another embodiment, the informational material can include instructions for identifying a suitable subject, e.g., a human, e.g., a human having, or at risk for a disorder characterized by excessive elastase activity. The informational material can include information about production of the compound, molecular weight of the compound, concentration, date of expiration, batch or production site information, and so forth. The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the compound and/or its use in the methods described herein. Of course, the informational material can also be provided in any combination of formats.

In addition to the compound, the composition of the kit can include other ingredients, such as a solvent or buffer, a stabilizer or a preservative, and/or a second agent for treating a condition or disorder described herein, e.g. a pulmonary (e.g., CF or COPD) or inflammatory (e.g., IBD or RA) disorder. Alternatively, the other ingredients can be included in the kit, but in different compositions or containers than the compound. In such embodiments, the kit can include instructions for admixing the compound and the other ingredients, or for using the compound together with the other ingredients.

The compound can be provided in any form, e.g., liquid, dried or lyophilized form. It is preferred that the compound be substantially pure and/or sterile. When the compound is provided in a liquid solution, the liquid solution preferably is an aqueous solution, with a sterile aqueous solution being preferred. When the compound is provided as a dried form, reconstitution generally is by the addition of a suitable solvent. The solvent, e.g., sterile water or buffer, can optionally be provided in the kit.

The kit can include one or more containers for the composition containing the compound. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the compound. For example, the kit includes a plurality of syringes, ampules, foil packets, or blister packs, each containing a single unit dose of the compound. The containers of the kits can be air tight, waterproof (e.g., impermeable to changes in moisture or evaporation), and/or light-tight.

In one embodiment wherein the compound contains a polypeptide that binds to an elastase, the instructions for diagnostic applications include the use of the compound to detect elastase, in vitro, e.g., in a sample, e.g., a biopsy or cells from a patient having a pulmonary disorder, or in vivo. In another embodiment, the instructions for therapeutic applications include suggested dosages and/or modes of administration in a patient with a pulmonary disorder. The kit can further contain a least one additional reagent, such as a diagnostic or therapeutic agent, e.g., a diagnostic or therapeutic agent as described herein, and/or one or more additional agents to treat the pulmonary disorder (e.g., another elastase inhibitor), formulated as appropriate, in one or more separate pharmaceutical preparations.

Treatments

A compound that contains (i) a polypeptide that includes a Kunitz domain and (ii) a moiety (such as a polymer) that increases the molecular weight of the compound has therapeutic and prophylactic utilities.

In one embodiment, the polypeptide includes a Kunitz domain or other inhibitor that inhibits an elastase, e.g., a neutrophil elastase. The compound can be administered to a subject to treat, prevent, and/or diagnose a variety of disorders, such as diseases characterized by unwanted or aberrant elastase activity. For example, the disease or disorder can be characterized by enhanced elastolytic activity of neutrophils. The disease or disorder may result from an increased neutrophil burden on a tissue, e.g., an epithelial tissue such as the epithelial surface of the lung. For example, the polypeptide that inhibit elastase can be used to treat or prevent pulmonary diseases such as cystic fibrosis (CF) or chronic obstructive pulmonary disorder (COPD), e.g., emphysema. The compound can also be administered to cells, tissues, or organs in culture, e.g. in vitro or ex vivo.

Polypeptides that include Kunitz domains that inhibit other proteases can be used to treat or prevent disorders associated with the activity of such other respective proteases.

As used herein, the term “treat” or “treatment” is defined as the application or administration of a compound that contains (i) apolypeptide that includes a Kunitz domain and (ii) a moiety (such as a polymer) that increases the molecular weight of the compound, alone or in combination with, a second agent to a subject, e.g., a patient, or application or administration of the agent to an isolated tissue or cell, e.g., cell line, from a subject, e.g., a patient, who has a disorder (e.g., a disorder as described herein), a symptom of a disorder or a predisposition toward a disorder, with the purpose to cure, heal, alleviate, relieve, alter, remedy, ameliorate, improve or affect the disorder, the symptoms of the disorder or the predisposition toward the disorder. Treating a cell refers to the inhibition, ablation, killing of a cell in vitro or in vivo, or otherwise reducing capacity of a cell, e.g., an aberrant cell, to mediate a disorder, e.g., a disorder as described herein (e.g., a pulmonary disorder). In one embodiment, “treating a cell” refers to a reduction in the activity and/or proliferation of a cell, e.g., a leukocyte or neutrophil. Such reduction does not necessarily indicate a total elimination of the cell, but a reduction, e.g., a statistically significant reduction, in the activity or the number of the cell.

As used herein, an amount of an elastase-binding compound effective to treat a disorder, or a “therapeutically effective amount” refers to an amount of the compound which is effective, upon single or multiple dose administration to a subject, in treating a subject, e.g., curing, alleviating, relieving or improving at least one symptom of a disorder in a subject to a degree beyond that expected in the absence of such treatment. For example, the disorder can be a pulmonary disorder, e.g., a pulmonary disorder described herein.

A “locally effective amount” refers to the amount (e.g., concentration) of the compound which is effective at detectably modulating activity of a target protein (e.g., elastase) in a tissue, e.g., in a region of the lung exposed to elastase, or a elastase-producing cell, such as a neutrophil. Evidence of modulation can include, e.g., increased amount of substrate, e.g., reduced proteolysis of the extracellular matrix.

As used herein, an amount of an elastase-binding compound effective to prevent a disorder, or a “a prophylactically effective amount” of the compound refers to an amount of an elastase-binding compound, e.g., a polypeptide-polymer compound described herein, which is effective, upon single- or multiple-dose administration to the subject, in preventing or delaying the occurrence of the onset or recurrence of a disorder, e.g., a pulmonary disorder.

The terms “induce”, “inhibit”, “potentiate”, “elevate”, “increase”, “decrease” or the like, e.g., which denote quantitative differences between two states, refer to a difference, e.g., a statistically significant difference (e.g., P<0.05, 0.02, or 0.005), between the two states.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

An exemplary, non-limiting range for a therapeutically or prophylactically effective amount of a compound described herein is 0.1-20 mg/kg, more preferably 1-10 mg/kg. The compound can be administered by intravenous infusion at a rate of less than 20, 10, 5, or 1 mg/min to reach a dose of about 1 to 50 mg/m² or about 5 to 20 mg/m². It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are only exemplary.

A pharmaceutical composition may include a “therapeutically effective amount” or a “prophylactically effective amount” of a compound described herein, e.g., a compound that includes a polypeptide that binds and inhibits a protease (e.g., elastase). A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the compound to elicit a desired response in the individual. A therapeutically effective amount is also one in which any toxic or detrimental effects of the composition is outweighed by the therapeutically beneficial effects. A “therapeutically effective dosage” preferably inhibits a measurable parameter, e.g., an increase in pulmonary function, relative to untreated subjects. The ability of a compound to inhibit a measurable parameter can be evaluated in an animal model system predictive of efficacy in a human disorder. Alternatively, this property of a composition can be evaluated by examining the ability of the compound to inhibit, such inhibition in vitro by assays known to the skilled practitioner, e.g., an assay described herein.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

As used herein, the term “subject” is intended to include human and non-human animals. The term “non-human animals” of the invention includes all vertebrates, e.g., non-mammals (such as chickens, amphibians, reptiles) and mammals, such as non-human primates, sheep, dog, cow, pig, etc.

In one embodiment, the subject is a human subject. Alternatively, the subject can be a non-human mammal expressing a human neutrophil elastase or an endogenous non-human neutrophil elastase protein or an elastase-like antigen to which an elastase-binding compound cross-reacts. A compound of the invention can be administered to a human subject for therapeutic purposes (discussed further below). Moreover, an elastase-binding compound can be administered to a non-human mammal expressing the elastase-like antigen to which the compound binds (e.g., a primate, pig or mouse) for veterinary purposes or as an animal model of human disease. Regarding the latter, such animal models may be useful for evaluating the therapeutic efficacy of the compound (e.g., testing of dosages and time courses of administration).

The subject method can be used on cells in culture, e.g. in vitro or ex vivo. The method can be performed on cells present in a subject, as part of an in vivo (e.g., therapeutic or prophylactic) protocol. For in vivo embodiments, the contacting step is effected in a subject and includes administering the elastase-binding compound to the subject under conditions effective to permit both binding of the compound to a target (e.g., an elastase) in the subject.

The compounds which inhibit elastase can reduce elastase-mediated degradation and its sequelae, such as persistent infection and inflammation, leading to destruction of tissue (e.g., destruction of airway epithelium).

Methods of administering compounds are described in “Pharmaceutical Compositions”. Suitable dosages of the compounds used will depend on the age and weight of the subject and the particular drug used. The compounds can be used as competitive agents to inhibit, reduce an undesirable interaction, e.g., between a natural or pathological agent and the elastase, e.g., between the extracellular matrix and elastase.

In one embodiment, the compounds are used to kill or ablate cells that express elastase in vivo. The compounds can be used by themselves or conjugated to an agent, e.g., a cytotoxic drug, radioisotope. This method includes: administering the compound alone or attached to a cytotoxic drug, to a subject requiring such treatment.

The terms “cytotoxic agent” and “cytostatic agent” refer to agents that have the property of inhibiting the growth or proliferation (e.g., a cytostatic agent), or inducing the killing of cells.

The compounds that include a polypeptide that includes a Kunitz domain and a moiety may also be used to deliver a variety of drugs including therapeutic drugs, a compound emitting radiation, molecules of plants, fungal, or bacterial origin, biological proteins, and mixtures thereof. For example, the Kunitz domain can be used to target the payload to a region of a subject which includes a protease that specifically interacts with the Kunitz domain.

Enzymatically active toxins and fragments thereof are exemplified by diphtheria toxin A fragment, nonbinding active fragments of diphtheria toxin, exotoxin A (from Pseudomonas aeruginosa), ricin A chain, abrin A chain, modeccin A chain, α-sacrin, certain Aleurites fordii proteins, certain Dianthin proteins, Phytolacca americana proteins (PAP, PAPII and PAP-S), Morodica charantia inhibitor, curcin, crotin, Saponaria officinalis inhibitor, gelonin, mitogillin, restrictocin, phenomycin, and enomycin. Procedures for preparing enzymatically active polypeptides of the immunotoxins are described in WO 84/03508 and WO 85/03508. Examples of cytotoxic moieties that can be conjugated to the antibodies include adriamycin, chlorambucil, daunomycin, methotrexate, neocarzinostatin, and platinum.

In the case of polypeptide toxins, recombinant nucleic acid techniques can be used to construct a nucleic acid that encodes the a polypeptide including a Kunitz domain and the cytotoxin (or a polypeptide component thereof) as translational fusions. The recombinant nucleic acid is then expressed, e.g., in cells and the encoded fusion polypeptide isolated. Then the fusion protein is physically associated with a moiety that increases the molecular weight of the compound, e.g., to stabilize half-life in vivo, and then attached to a moiety (e.g., a polymer).

Procedures for conjugating proteins with the cytotoxic agents have been previously described. For conjugating chlorambucil with proteins, see, e.g., Flechner (1973) European Journal of Cancer, 9:741-745; Ghose et al. (1972) British Medical Journal, 3:495-499; and Szekerke, et al. (1972) Neoplasma, 19:211-215. For conjugating daunomycin and adriamycin to proteins, see, e.g., Hurwitz, E. et al. (1975) Cancer Research, 35:1175-1181 and Amon et al. (1982) Cancer Surveys, 1:429-449. For preparing protein-ricin conjugates, see, e.g., U.S. Pat. No. 4,414,148 and by Osawa, T., et al. (1982) Cancer Surveys, 1:373-388 and the references cited therein. Coupling procedures as also described in EP 226 419.

Also encompassed by the present invention is a method of killing or ablating which involves using the compound for prophylaxis. For example, these materials can be used to prevent or delay development or progression of a lung disease.

Use of the therapeutic methods of the present invention to treat lung diseases has a number of benefits. Since the polypeptide portion of the compound specifically recognizes elastase, other tissue is spared and high levels of the agent are delivered directly to the site where therapy is required. Treatment in accordance with the present invention can be effectively monitored with clinical parameters. Alternatively, these parameters can be used to indicate when such treatment should be employed.

Pulmonary Disorders and Methods and Formulations Therefor

hNE inhibitor polypeptides that are physically associated with a moiety (e.g., a polymer) can be used to treat pulmonary disorders such as emphysema, cystic fibrosis, COPD, bronchitis, pulmonary hypertension, acute respiratory distress syndrome, interstitial lung disease, asthma, smoke intoxication, bronchopulmonary dysplasia, pneumonia, thermal injury, and lung transplant rejection.

Cystic Fibrosis. Cystic fibrosis (CF) is a genetic disease affecting approximately 30,000 children and adults in the United States. A defect in the CF gene causes the body to produce an abnormally thick, sticky mucus that clogs the lungs and leads to life-threatening lung infections. A diagnostic for the genetic disorder includes a sweat test which can include measuring chloride concentration in sweat collected on gauze or filter paper, measuring sodium concentration in sweat collected on gauze or filter paper, and pilocarpine delivery and current density in sweat collection. The gene that causes CF has been identified and a number of mutations in the gene are known.

In one embodiment, a hNE inhibitor polypeptide that is physically associated with a moiety (e.g., a polymer) is used to ameliorate at least one symptom of CF, e.g., to reduce pulmonary lesions in the lungs of a CF patient.

This compound can also be used to ameliorate at least one symptom of a chronic obstructive pulmonary disease (COPD). Emphysema, along with chronic bronchitis, is part of chronic obstructive pulmonary disease (COPD). It is a serious lung disease and is progressive, usually occurring in elderly patients. COPD causes over-inflation of structures in the lungs known as alveoli or air sacs. The walls of the alveoli break down resulting in a decrease in the respiratory ability of the lungs. Patients with this disease may first experience shortness of breath and cough. One clinical index for evaluating COPD is the destructive index which measures a measure of alveolar septal damage and emphysema, and has been proposed as a sensitive index of lung destruction that closely reflects functional abnormalities, especially loss of elastic recoil. See, e.g., Am Rev Respir Dis 1991 July; 144(1):156-9. The compound can be used to reduce the destructive index in a patient, e.g., a statistically significant amount, e.g., at least 10, 20, 30, or 40% or at least to within 50, 40, 30, or 20% of normal of a corresponding age and gender-matched individual.

In one aspect, the invention provides a composition that includes an hNE inhibitor polypeptide that is physically associated with a moiety for treatment of a pulmonary disorder (e.g., cystic fibrosis, COPD). The composition can be formulated for inhalation or other mode of pulmonary delivery. Accordingly, the compounds described herein can be administered by inhalation to pulmonary tissue. The term “pulmonary tissue” as used herein refers to any tissue of the respiratory tract and includes both the upper and lower respiratory tract, except where otherwise indicated. A hNE inhibitor polypeptide that is physically associated with a moiety (e.g., a polymer) can be administered in combination with one or more of the existing modalities for treating pulmonary diseases.

In one example the compound is formulated for a nebulizer. In one embodiment, the compound can be stored in a lyophilized form (e.g., at room temperature) and reconstituted in solution prior to inhalation.

It is also possible to formulate the compound for inhalation using a medical device, e.g., an inhaler. See, e.g., U.S. Pat. No. 6,102,035 (a powder inhaler) and U.S. Pat. No. 6,012,454 (a dry powder inhaler). In one embodiment, the inhaler is a metered dose inhaler.

The three common systems used to deliver drugs locally to the pulmonary air passages include dry powder inhalers (DPIs), metered dose inhalers (MDIs) and nebulizers. MDIs, the most popular method of inhalation administration, may be used to deliver medicaments in a solubilized form or as a dispersion. Typically MDIs comprise a Freon or other relatively high vapor pressure propellant that forces aerosolized medication into the respiratory tract upon activation of the device. Unlike MDIs, DPIs generally rely entirely on the inspiratory efforts of the patient to introduce a medicament in a dry powder form to the lungs. Nebulizers form a medicament aerosol to be inhaled by imparting energy to a liquid solution. Direct pulmonary delivery of drugs during liquid ventilation or pulmonary lavage using a fluorochemical medium has also been explored. These and other methods can be used to deliver a hNE inhibitor polypeptide that is physically associated with a moiety (e.g., a polymer).

For example, for administration by inhalation, the hNE inhibitor polypeptides that is physically associated with a moiety (e.g., a polymer) are delivered in the form of an aerosol spray from pressured container or dispenser which contains a suitable propellant or a nebulizer. The compound may be in the form of a dry particle or as a liquid. Particles that include the compound can be prepared, e.g., by spray drying, by drying an aqueous solution of the hNE inhibitor polypeptide that is physically associated with a moiety (e.g., a polymer) with a charge neutralizing agent and then creating particles from the dried powder or by drying an aqueous solution in an organic modifier and then creating particles from the dried powder.

The compound may be conveniently delivered in the form of an aerosol spray presentation from pressurized packs or a nebulizer, with the use of a suitable propellant, e.g., dichlorodifluoromethane, trichlorofluoromethane, dielilorotetrafluoroctliane, carbon dioxide or other suitable gas. In the case of a pressurized aerosol, the dosage unit may be determined by providing a valve to deliver a metered amount. Capsules and cartridges for use in an inhaler or insufflator may be formulated containing a powder mix of the a hNE inhibitor polypeptide that is physically associated with a moiety (e.g., a polymer) and a suitable powder base such as lactose or starch, if the particle is a formulated particle. In addition to the formulated or unformulated compound, other materials such as 100% DPPC or other surfactants can be mixed with the hNE inhibitor polypeptide that is physically associated with a moiety (e.g., a polymer) to promote the delivery and dispersion of formulated or unformulated compound. Methods of preparing dry particles are described, for example, in PCT Publication WO 02/32406.

The a hNE inhibitor polypeptide that is physically associated with a moiety (e.g., a polymer), e.g., as dry aerosol particles, when administered can be rapidly absorbed and can produce a rapid local or systemic therapeutic result. Administration can be tailored to provide detectable activity within 2 minutes, 5 minutes, 1 hour, or 3 hours of administration. In some embodiments, the peak activity can be achieved even more quickly, e.g., within one half hour or even within ten minutes. Alternatively, a hNE inhibitor polypeptide that is physically associated with a moiety (e.g., a polymer) can be formulated for longer biological half-life can be used as an alternative to other modes of administration, e.g., such that the compound enters circulation from the lung and is distributed to other organs or to a particular target organ.

In one embodiment, the hNE inhibitor polypeptide that is physically associated with a moiety (e.g., a polymer) is delivered in an amount such that at least 5% of the mass of the polypeptide is delivered to the lower respiratory tract or the deep lung. Deep lung has an extremely rich capillary network. The respiratory membrane separating capillary lumen from the alveolar air space is very thin (≦6 μm) and extremely permeable. In addition, the liquid layer lining the alveolar surface is rich in lung surfactants. In other embodiments, at least 2%, 3%, 5%, 10%, 20%, 30%, 40%, 50%, 60%, 70%, or 80% of the composition of a hNE inhibitor polypeptide that is physically associated with a moiety (e.g., a polymer) is delivered to the lower respiratory tract or to the deep lung. Delivery to either or both of these tissues results in efficient absorption of the compound and high bioavailability. In one embodiment, the compound is provided in a metered dose using, e.g., an inhaler or nebulizer. For example, the compound is delivered in a dosage unit form of at least about 0.02, 0.1, 0.5, 1, 1.5, 2, 5, 10, 20, 40, or 50 mg/puff or more.

The percent bioavailability can be calculated as follows: the percent bioavailability=(AUCₙₒₙ₋ᵢₙᵥₐₛᵢᵥₑ/AUC_{i.v. or s.c.})×(dose_{i.v. or s.c.}/doseₙₒₙ₋ᵢₙᵥₐₛᵢᵥₑ)×100.

Although not necessary, delivery enhancers such as surfactants can be used to further enhance pulmonary delivery. A “surfactant” as used herein refers to a compound having a hydrophilic and lipophilic moiety, which promotes absorption of a drug by interacting with an interface between two immiscible phases. Surfactants are useful in the dry particles for several reasons, e.g., reduction of particle agglomeration, reduction of macrophage phagocytosis, etc. When coupled with lung surfactant, a more efficient absorption of the compound can be achieved because surfactants, such as DPPC, will greatly facilitate diffusion of the compound. Surfactants are well known in the art and include but are not limited to phosphoglycerides, e.g., phosphatidylcholines, L-alpha-phosphatidylcholine dipalmitoyl (DPPC) and diphosphatidyl glycerol (DPPG); hexadecanol; fatty acids; polyethylene glycol (PEG); polyoxyethylene-9-; auryl ether; palmitic acid; oleic acid; sorbitan trioleate (Span 85); glycocholate; surfactin; poloxomer; sorbitan fatty acid ester; sorbitan trioleate; tyloxapol; and phospholipids.

IBD and Methods and Formulations Therefor

In one embodiment, a hNE inhibitor polypeptide that includes a Kunitz domain that inhibits elastase and is physically associated with a moiety (e.g., a polymer) that increases the molecular weight of the compound is used to ameliorate at least one symptom of an inflammatory bowel disease, e.g., ulcerative colitis or Crohn's disease.

Inflammatory bowel diseases (IBD) are generally chronic, relapsing intestinal inflammation. IBD refers to two distinct disorders, Crohn's disease and ulcerative colitis (UC). Both diseases may involve either a dysregulated immune response to GI tract antigens, a mucosal barrier breach, and/or an adverse inflammatory reaction to a persistent intestinal infection (see, e.g., MacDermott, R. P., J Gastroenterology, 31:907:-916 (1996)).

In patients with IBD, ulcers and inflammation of the inner lining of the intestines lead to symptoms of abdominal pain, diarrhea, and rectal bleeding. Ulcerative colitis occurs in the large intestine, while in Crohn's, the disease can involve the entire GI tract as well as the small and large intestines. For most patients, IBD is a chronic condition with symptoms lasting for months to years. The clinical symptoms of IBD are intermittent rectal bleeding, crampy abdominal pain, weight loss and diarrhea. Diagnosis of IBD is based on the clinical symptoms, the use of a barium enema, but direct visualization (sigmoidoscopy or colonoscopy) is the most accurate test.

Symptoms of IBD include, for example, abdominal pain, diarrhea, rectal bleeding, weight loss, fever, loss of appetite, and other more serious complications, such as dehydration, anemia and malnutrition. A number of such symptoms are subject to quantitative analysis (e.g. weight loss, fever, anemia, etc.). Some symptoms are readily determined from a blood test (e.g. anemia) or a test that detects the presence of blood (e.g. rectal bleeding). A clinical index can also be used to monitor IBD such as the Clinical Activity Index for Ulcerative Colitis. See also, e.g., Walmsley et al. Gut. 1998 July; 43(1):29-32 and Jowett et al. (2003) Scand J Gastroenterol. 38(2):164-71.

In one embodiment, administration of the compound to a subject having or predisposed to having ulcerative colitis causes amelioration of the index, e.g., a statistically significant change in the index. The compound includes hNE inhibitor polypeptide that is physically associated with a moiety (e.g., a hydrophilic polymer)

In one embodiment, administration of the compound to a subject having or predisposed to having IBD causes amelioration of at least one symptom of IBD.

Crohn's disease, an idiopathic inflammatory bowel disease, is characterized by chronic inflammation at various sites in the gastrointestinal tract. While Crohn's disease most commonly affects the distal ileum and colon, it may manifest itself in any part of the gastrointestinal tract from the mouth to the anus and perianal area. The prognosis and diagnosis of Crohn's disease can be measured using a clinical index, e.g., Crohn's Disease Activity Index. See, e.g., American Journal of Natural Medicine, July/August 1997, and Best W R, et al., “Development of a Crohn's disease activity index.” Gastroenterology 70:439-444, 1976. In one embodiment, administration of the compound to a subject having or predisposed to having Crohn's disease causes amelioration of the index, e.g., a statistically significant change in the index, or amelioration of at least one symptom of Crohn's disease.

Accordingly, in one aspect, the invention provides a composition that includes an hNE inhibitor polypeptide for treatment of a bowel disease (e.g., a colitis such as ulcerative colitis, Crohn's disease or IBP) or other gastrointestinal or rectal disease. The hNE inhibitor polypeptide includes a Kunitz domain that inhibits elastase and is physically associated with a moiety (e.g., a polymer) that increases the molecular weight of the compound. The composition can be formulated as a suppository.

Suppositories can be formulated with base ingredients such as waxes, oils, and fatty alcohols with characteristics of remaining in solid state at room temperatures and melting at body temperatures. The active ingredients of this invention with or without optional therapeutic ingredients, like hydrocortisone (1.0%), topical anesthetics like benzocaine (1.0 to 6.0%) or others as already listed may be prepared at appropriate pH values; for example pH 5 liquid fatty alcohols, such as oleyl alcohol (range 45% to 65%) or solid higher fatty alcohols like cetyl or stearyl alcohol (30% to 50%). The base ingredients are well known in the art of this industry. See, e.g., U.S. Pat. Nos. 4,945,084 and 5,196,405.

The composition may also be used as an active ingredient in creams, lotions, ointments, sprays, pads, patches, enemas, foams and suppositories and others or in delivery vehicles such as micro-encapsulation in liposomes or glycospheres. Other delivery technologies include microsponges or the substitute cell membrane (Completech™) which entrap the active ingredients for both protection and for slower release. Rectal foams can be prepared as topical aerosol compositions may also be used, e.g., to treat (ulcerative colitis, Crohns colitis, and others).

Diagnostic Uses

A compound that contains (i) a polypeptide that includes a Kunitz domain and (ii) a moiety (such as a polymer) that increases the molecular weight of the compound also has diagnostic utilities.

In one aspect, the present invention provides a diagnostic method for detecting the presence of a elastase protein, in vitro (e.g., a biological sample, such as tissue, biopsy or in vivo (e.g., in vivo imaging in a subject). The method includes: (i) contacting a sample with an compound comprising a polypeptide and a polymer, wherein the polypeptide comprises a Kunitz domain, and wherein the Kunitz domain binds an elastase; and (ii) detecting formation of a complex between the elastase ligand and the sample. The method can also include contacting a reference sample (e.g., a control sample) with the ligand, and determining the extent of formation of the complex between the ligand and the sample relative to the same for the reference sample. A change, e.g., a statistically significant change, in the formation of the complex in the sample or subject relative to the control sample or subject can be indicative of the presence of elastase in the sample.

Another method includes: (i) administering the compound to a subject; and (iii) detecting formation of a complex between the compound, and the target elastase. The detecting can include determining location or time of formation of the complex.

The compound can be directly or indirectly labeled with a detectable substance to facilitate detection of the bound or unbound antibody. Suitable detectable substances include various enzymes, prosthetic groups, fluorescent materials, luminescent materials and radioactive materials.

Complex formation between the compound and elastase can be detected by measuring or visualizing either the ligand bound to the elastase or unbound ligand. Conventional detection assays can be used, e.g., an enzyme-linked immunosorbent assays (ELISA), a radioimmunoassay (RIA) or tissue immunohistochemistry. Further to labeling the compound, the presence of elastase can be assayed in a sample by a competition immunoassay utilizing standards labeled with a detectable substance and an unlabeled elastase ligand. In one example of this assay, the biological sample, the labeled standards and the compound are combined and the amount of labeled standard bound to the unlabeled ligand is determined. The amount of elastase in the sample is inversely proportional to the amount of labeled standard bound to the compound.

Fluorophore and chromophore labeled protein ligands can be prepared. A variety of suitable fluorescers and chromophores are described by Stryer (1968) Science, 162:526 and Brand, L. et al. (1972) Annual Review of Biochemistry, 41:843-868. The protein ligands can be labeled with fluorescent chromophore groups by conventional procedures such as those disclosed in U.S. Pat. Nos. 3,940,475, 4,289,747, and 4,376,110. One group of fluorescers having a number of the desirable properties described above is the xanthene dyes, which include the fluoresceins and rhodamines. Another group of fluorescent compounds are the naphthylamines. Once labeled with a fluorophore or chromophore, the protein ligand can be used to detect the presence or localization of the elastase in a sample, e.g., using fluorescent microscopy (such as confocal or deconvolution microscopy).

Protein Arrays. The compound can also be immobilized on a protein array. The protein array can be used as a diagnostic tool, e.g., to screen medical samples (such as isolated cells, blood, sera, biopsies, and the like). Methods of producing polypeptide arrays are described, e.g., above.

In vivo Imaging. In still another embodiment, the invention provides a method for detecting the presence of elastase or an elastase-expressing tissue in vivo. The method includes (i) administering to a subject (e.g., a patient having a pulmonary or respiratory disorder) an elastase-binding compound, conjugated to a detectable marker; (ii) exposing the subject to a means for detecting said detectable marker to the elastase-expressing tissues or cells. For example, the subject is imaged, e.g., by NMR or other tomographic means.

Examples of labels useful for diagnostic imaging in accordance with the present invention include radiolabels such as ¹³¹I, ¹¹¹In, ¹²³I, ^{99m}Tc, ³²P, ¹²⁵I, ³H, ¹⁴C, and ¹⁸⁸Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed. The elastase-binding compound can be labeled with such reagents using known techniques. For example, see Wensel and Meares (1983) Radioimmunoimaging and Radioimmunotherapy, Elsevier, New York for techniques relating to the radiolabeling of proteins and D. Colcher et al. (1986) Meth. Enzymol. 121: 802-816.

A radiolabeled compound of this invention can also be used for in vitro diagnostic tests. The specific activity of an isotopically-labeled compound depends upon the half-life, the isotopic purity of the radioactive label, and how the label is incorporated into the compound.

Procedures for labeling polypeptides (e.g., the polypeptide portion of the compound) with the radioactive isotopes (such as ¹⁴C, ³H, ³⁵S, ¹²⁵I, ³²P, ¹³¹I) are generally known. For example, tritium labeling procedures are described in U.S. Pat. No. 4,302,438. Iodinating, tritium labeling, and ³⁵S labeling procedures, e.g., as adapted for murine monoclonal antibodies, are described, e.g., by Goding, J. W. (Monoclonal antibodies: principles and practice: production and application of monoclonal antibodies in cell biology, biochemistry, and immunology 2nd ed. London; Orlando: Academic Press, 1986. pp 124-126) and the references cited therein. Other procedures for iodinating polypeptides, are described by Hunter and Greenwood (1962) Nature 144:945, David et al. (1974) Biochemistry 13:1014-1021, and U.S. Pat. Nos. 3,867,517 and 4,376,110. Radiolabeling elements which are useful in imaging include ¹²³I, ¹³¹I, ¹¹¹In, and ^{99m}Tc, for example. Procedures for iodinating polypeptides are described by Greenwood, F. et al. (1963) Biochem. J. 89:114-123; Marchalonis, J. (1969) Biochem. J. 113:299-305; and Morrison, M. et al. (1971) Immunochemistry 289-297. Procedures for ^{99m}Tc-labeling are described by Rhodes, B. et al. in Burchiel, S. et al. (eds.), Tumor Imaging: The Radioimmunochemical Detection of Cancer, New York: Masson 111-123 (1982) and the references cited therein. Procedures suitable for ¹¹¹In-labeling antibodies are described by Hnatowich, D. J. et al. (1983) J. Immul. Methods, 65:147-157, Hnatowich, D. et al. (1984) J. Applied Radiation, 35:554-557, and Buckley, R. G. et al. (1984) F.E.B.S. 166:202-204.

In the case of a radiolabeled compound, the compound is administered to the patient, is localized to the tissue the antigen with which the compound interacts, and is detected or “imaged” in vivo using known techniques such as radionuclear scanning using e.g., a gamma camera or emission tomography. See e.g., A. R. Bradwell et al., “Developments in Antibody Imaging”, Monoclonal Antibodies for Cancer Detection and Therapy, R. W. Baldwin et al., (eds.), pp 65-85 (Academic Press 1985). Alternatively, a positron emission transaxial tomography scanner, such as designated Pet VI located at Brookhaven National Laboratory, can be used where the radiolabel emits positrons (e.g., ¹¹C, ¹⁸F, ¹⁵O, and ¹³N).

MRI Contrast Agents. Magnetic Resonance Imaging (MRI) uses NMR to visualize internal features of living subject, and is useful for prognosis, diagnosis, treatment, and surgery. MRI can be used without radioactive tracer compounds for obvious benefit. Some MRI techniques are summarized in EP-A-0 502 814. Generally, the differences related to relaxation time constants T1 and T2 of water protons in different environments is used to generate an image. However, these differences can be insufficient to provide sharp high resolution images.

The differences in these relaxation time constants can be enhanced by contrast agents. Examples of such contrast agents include a number of magnetic agents paramagnetic agents (which primarily alter T1) and ferromagnetic or superparamagnetic (which primarily alter T2 response). Chelates (e.g., EDTA, DTPA and NTA chelates) can be used to attach (and reduce toxicity) of some paramagnetic substances (e.g., Fe⁺³, Mn⁺², Gd⁺³). Other agents can be in the form of particles, e.g., less than 10 μm to about 10 nM in diameter). Particles can have ferromagnetic, antiferromagnetic or superparamagnetic properties. Particles can include, e.g., magnetite (Fe₃O₄), γ-Fe₂O₃, ferrites, and other magnetic mineral compounds of transition elements. Magnetic particles may include: one or more magnetic crystals with and without nonmagnetic material. The nonmagnetic material can include synthetic or natural polymers (such as sepharose, dextran, dextrin, starch and the like.

The compounds can also be labeled with an indicating group containing of the NMR-active ¹⁹F atom, or a plurality of such atoms inasmuch as (i) substantially all of naturally abundant fluorine atoms are the ¹⁹F isotope and, thus, substantially all fluorine-containing compounds are NMR-active; (ii) many chemically active polyfluorinated compounds such as trifluoracetic anhydride are commercially available at relatively low cost, and (iii) many fluorinated compounds have been found medically acceptable for use in humans such as the perfluorinated polyethers utilized to carry oxygen as hemoglobin replacements. After permitting such time for incubation, a whole body MRI is carried out using an apparatus such as one of those described by Pykett (1982) Scientific American, 246:78-88 to locate and image cancerous tissues.

Also within the scope of the invention are kits comprising the compound that binds to elastase and instructions for use, e.g., the use of the compound (e.g., comprising an elastase-binding polypeptide and a polymer to detect elastase, in vitro, e.g., in a sample, e.g., a biopsy or cells from a patient having a pulmonary disorder, or in vivo, e.g., by imaging a subject. The kit can further contain a least one additional reagent, such as a label or additional diagnostic agent. For in vivo use the compound can be formulated as a pharmaceutical composition.

An exemplary amino acid sequence of a human neutrophil elastase:

(Also listed in GenBank® under: gi|4503549|ref|NP_{—}001963.1| elastase 2, neutrophil [Homo sapiens])

MTLGRRLACLFLACVLPALLLGGTALASEIVGGRRARPHAWPFMVSLQLR
GGHFCGATLIAPNFVMSAAHCVANVNVRAVRVVLGAHNLSRREPTRQVFA
VQRIFENGYDPVNLLNDIVILQLNGSATINANVQVAQLPAQGRRLGNGVQ
CLAMGWGLLGRNRGIASVLQELNVTVVTSLCRRSNVCTLVRGRQAGVCFG
DSGSPLVCNGLIHGIASFVRGGCASGLYPDAFAPVAQFVNWIDSIIQRSE
DNPCPHPRDPDPASRTH

An exemplary nucleotide sequence of a human neutrophil elastase:

(Also listed in GenBank® under: gi|4503548|ref|NM_{—}001972.1| Homo sapiens elastase 2, neutrophil (ELA2), mRNA)

GCACGGAGGGGCAGAGACCCCGGAGCCCCAGCCCCACCATGACCCTCGGC
CGCCGACTCGCGTGTCTTTTCCTCGCCTGTGTCCTGCCGGCCTTGCTGCT
GGGGGGCACCGCGCTGGCCTCGGAGATTGTGGGGGGCCGGCGAGCGCGGC
CCCACGCGTGGCCCTTCATGGTGTCCCTGCAGCTGCGCGGAGGCCACTTC
TGCGGCGCCACCCTGATTGCGCCCAACTTCGTCATGTCGGCCGCGCACTG
CGTGGCGAATGTAAACGTCCGCGCGGTGCGGGTGGTCCTGGGAGCCCATA
ACCTCTCGCGGCGGGAGCCCACCCGGCAGGTGTTCGCCGTGCAGCGCATC
TTCGAAAACGGCTACGACCCCGTAAACTTGCTCAACGACATCGTGATTCT
CCAGCTCAACGGGTCGGCCACCATCAACGCCAACGTGCAGGTGGCCCAGC
TGCCGGCTCAGGGACGCCGCCTGGGCAACGGGGTGCAGTGCCTGGCCATG
GGCTGGGGCCTTCTGGGCAGGAACCGTGGGATCGCCAGCGTCCTGCAGGA
GCTCAACGTGACGGTGGTGACGTCCCTCTGCCGTCGCAGCAACGTCTGCA
CTCTCGTGAGGGGCCGGCAGGCCGGCGTCTGTTTCGGGGACTCCGGCAGC
CCCTTGGTCTGCAACGGGCTAATCCACGGAATTGCCTCCTTCGTCCGGGG
AGGCTGCGCCTCAGGGCTCTACCCCGATGCCTTTGCCCCGGTGGCACAGT
TTGTAAACTGGATCGACTCTATCATCCAACGCTCCGAGGACAACCCCTGT
CCCCACCCCCGGGACCCGGACCCGGCCAGCAGGACCCACTGAGAAGGGCT
GCCCGGGTCACCTCAGCTGCCCACACCCACACTCTCCAGCATCTGGCACA
ATAAACATTCTCTGTTTTGT

The following non-limiting examples further illustrate aspects of the invention:

### EXAMPLES

Peptides and small proteins are rapidly cleared from circulation in vivo. The rapid clearance often greatly limits therapeutic potency. High doses and frequent administration are needed to achieve therapeutic effects. DX-890 consists of 56 amino acids, contains three intramolecular disulfide bonds, and has a molecular weight of 6,237 Da. Use of MPEG succinimidyl propionic acid (see below) at pH 6 can be used to preferentially couple to the N-terminus.

Unmodified DX-890 is a small protein.

In mice, the clearance of unmodified DX-890 from circulation is so fast that at 30 minutes after injection less than 20% of the material remains in circulation. Clearance from circulation in larger mammals (such as humans) may also be rapid.

The addition of a single PEG 20 KDa or 30 KDa moiety to DX-890 greatly increases the in vivo circulating half-life of the compound when it is delivered intravenously. In mice, the in vivo half-life for mono-PEGylated DX-890 is increased at least 5- to 10-fold (depending on the PEG used). In rabbits, the 30 KDa mono-PEGylated DX-890 shows at least a 25- to 100-fold increase in in vivo half-life (to about 3 days) relative to unmodified DX-890. The improvements in DX-890 circulatory half-life can allow lower doses and/or less frequent administrations for therapeutic uses.

### Example 1

### Preparation of PEGylated DX-890

Materials

Native DX-890 (Mw=6,37 Da, Dyax, lot# BBG2016/9 HIC2, 4.46 mg/ml)

MSPA20K (Mw-21,600, Nektar Therapeutics, Lot# PT-O5C-11)

MSPA3OK (Mn=31,300, Nektar Therapeutics, Lot# PT-O5C-12)

Sodium phosphate monobasic, monohydrate (Fisher, F W 137.99, lot# 015507B)

Sodium phosphate dibasic, anhydrous (E M Science, FW 141.96, lot# 127074-1 17617)

Sodium hydroxide, certified A.C.S., (Fisher, F W 40.00, lot# 995312)

Sodium chloride, certified A.C.S. (Fisher Scientific)

Tris/glycine/SDS, 10×, protein electrophoresis buffer (Bio-Rad)

Laemmli sample buffer (Bio-Rad)

SigmaMarker, low range (M.W. 6,000)(Sigma)

SigmaMarker, high range (M.W. 36,000)(Sigma)

10% Tris-HCl ready gel (10well, 30 ul, Bio-Rad)

GelCode blue stain reagent (Pierce)

Analytical Methods

SDS-PAGE Analysis. Samples were characterized by sodium dodecyl sulfate-polyacrylamide gel electrophoresis (SDS-PAGE). After 20 ul of each sample were mixed with 20 ul of Laemmli sample buffer, each sample mixture was heated for 5 min in a boiling water bath. Then, each sample was loaded onto a 10% Tris-HCl ready gel, which was run for 30 minutes at 200V with Tris/glycine/SDS electrophoresis buffer using Mini-PROTEIN 3 Precast Gel Electrophoresis System manufactured by Bio-Rad.

PEGylation and Purification of DX-890

1. Random-Site Modification

1.1 PEGylation of DX-890 with MPEG-SPA20K at pH7.4

DX-890 (4.46 mg/ml stock solution) was reacted with MPEG-SPA20K at pH7.4. After quenching the reaction, the PEGylated reaction mixture was stored at −20° C. until purification.

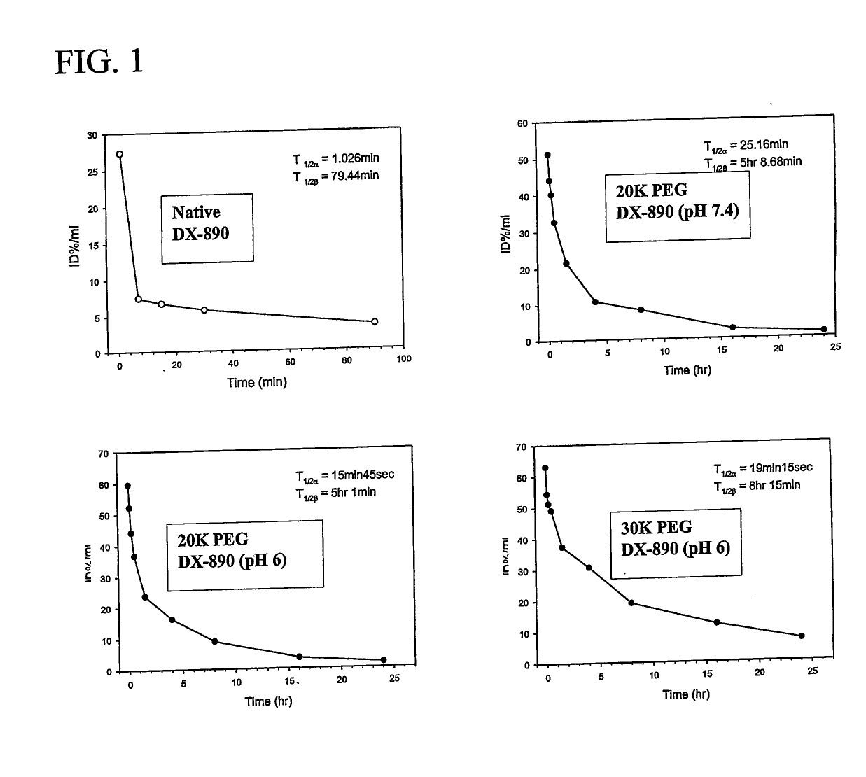

After thawing the PEGylated reaction mixture, the solution was dialyzed overnight. The next day, the crude PEGylated protein was loaded onto an ion exchange column. The fractions from 7-15, which contained pure mono-PEGylated DX-890, were collected (FIG. 1) and were then dialyzed in 5 mM sodium phosphate buffer (pH6.0), containing 100 mM NaCl. Following the dialysis, the mono-PEGylated protein was concentrated using Centricon-10 at −4° C. by spinning the solution in the centrifuge at 4900 μg.

The concentrated protein was filtered through a 0.22 um pore size syringe filter. The mono-PEGylated protein was stored at −20° C.

1.2 PEGylation of DX-890 with MPEG-SPA30K at pH7.4

DX-890 (4.46 mg/ml stock solution) was reacted with MPEG-SPA30K at pH7.4. After quenching the reaction, the PEGylated reaction mixture was stored at −20° C. until purification.

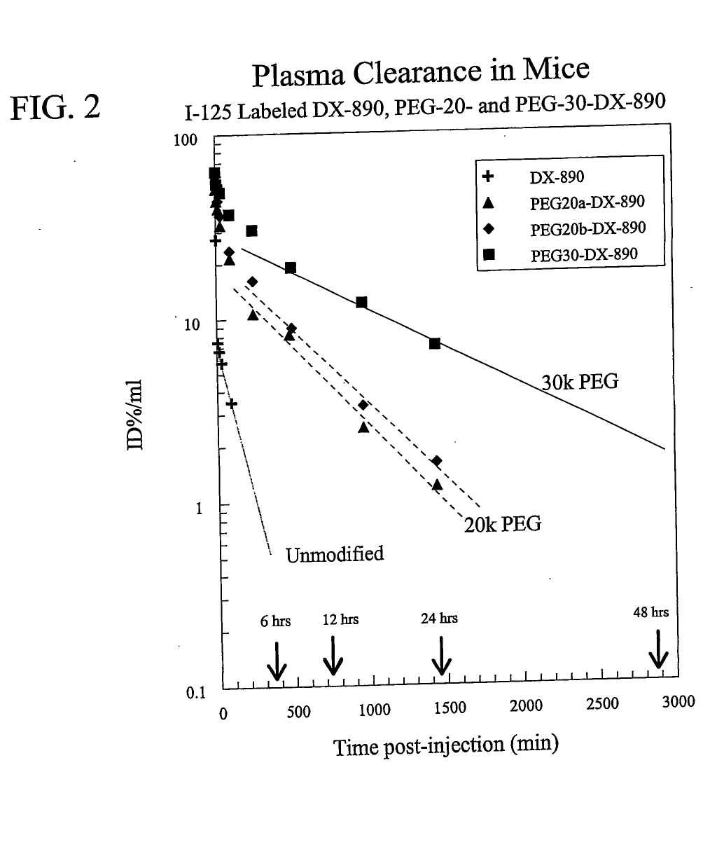

After thawing the PEGylated reaction mixture, the solution was dialyzed overnight. The next day, the crude PEGylated protein was loaded onto an ion exchange column. The fractions from 18-24, which contained pure mono-PEGylated DX-890, were collected (FIG. 2) and were then dialyzed in 5 mM sodium phosphate buffer (pH6.0), containing 100 mM NaCl. Following the dialysis, the mono-PEGylated protein was concentrated using Centricon-10 at −4° C. by spinning the solution in the centrifuge at 4900 χg.

The concentrated protein was filtered through a 0.22 um pore size syringe filter. The mono-PEGylated protein was stored at −20° C.

2. N-Terminal-Site Modification

2.1 PEGylation of DX-890 with MPEG-SPA20K at pH6.0

DX-890 (4.46 mg/ml stock solution) was reacted with MPEG-SPA20K at pH6.0. After quenching the reaction, the PEGylated reaction mixture was stored at −20° C. until purification.

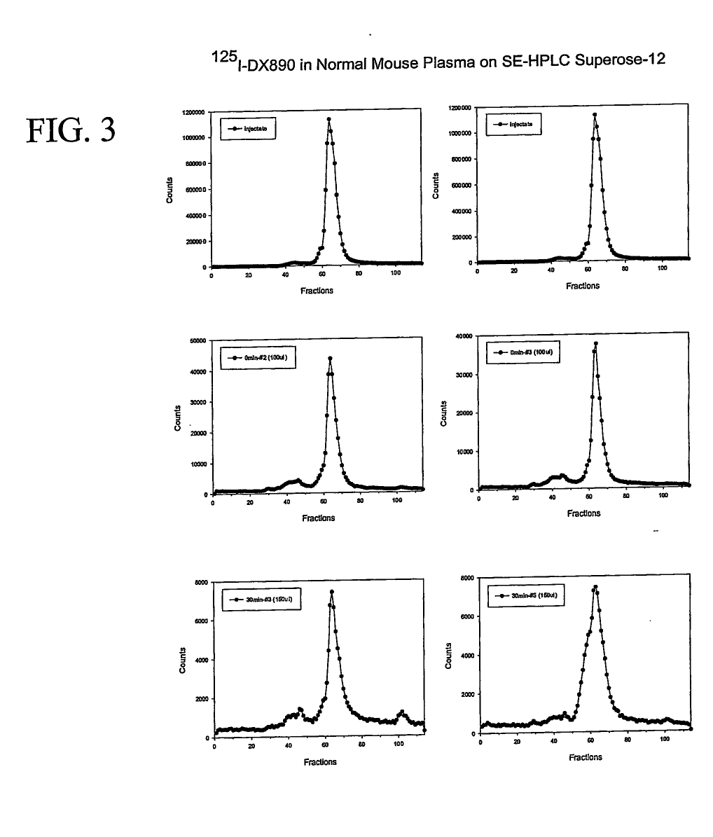

After thawing the PEGylated reaction mixture, the solution was dialyzed overnight. The next day, the crude PEGylated protein was loaded onto an ion exchange column. The fractions from 7-13, which contained pure mono-PEGylated DX-890, were collected (FIG. 3) and were then dialyzed in 5 mM sodium phosphate buffer (pH6.0), containing 100 mM NaCl. Following the dialysis, the mono-PEGylated protein was concentrated using Centricon-10 at −4° C. by spinning the solution in the centrifuge at 4900 χg.

The concentrated protein was filtered through 0.22 um pore size syringe filter. The mono-PEGylated protein was stored at −20° C.

2.2 PEGylation of DX-890 with MPEG-SPA30K at pH6.0

DX-890 (4.46 mg/ml stock solution) was reacted MPEG-SPA30K at pH6.0. After quenching the reaction, the PEGylated reaction mixture was stored at −20° C. until purification.

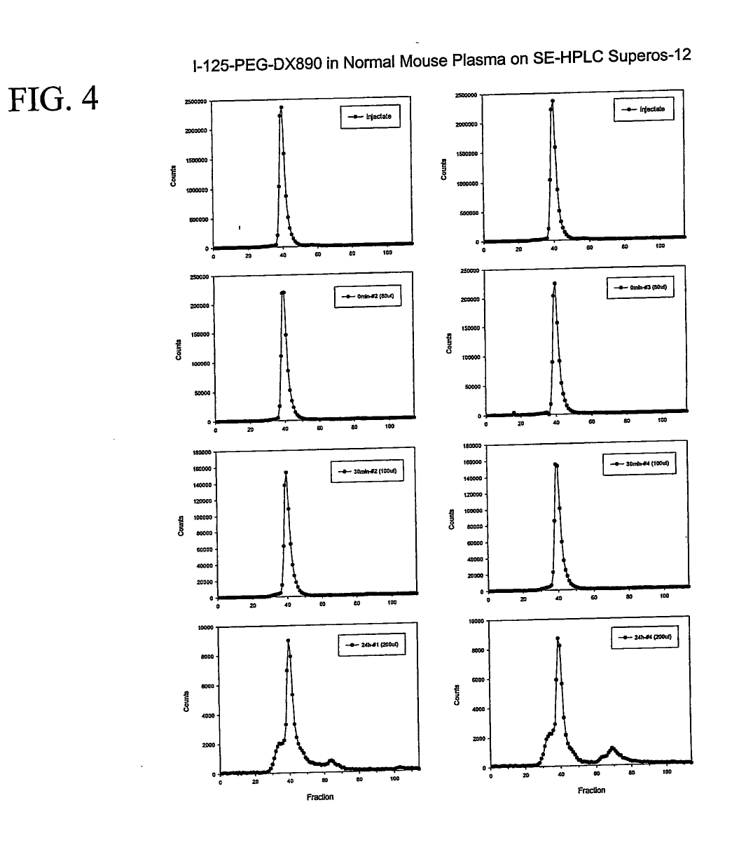

After thawing the PEGylated reaction mixture, the solution was dialyzed overnight. The next day, the crude PEGylated protein was loaded onto an ion exchange column. The fractions from 7-15, which contained pure mono-PEGylated DX-890, were collected (FIG. 4) and were then dialyzed in 5 mM sodium phosphate buffer (pH6.0) containing 100 mM NaCl. Following the dialysis, the mono-PEGylated protein was concentrated using Centricon-10 at −4° C. by spinning the solution in the centrifuge at 4900 χg.

The concentrated protein was filtered through a 0.22 um pore size syringe filter. The mono-PEGylated protein was stored at −20° C.

For each of the PEGylated DX-890 preparations, SDS-PAGE analysis shows that the sample is composed of virtually all mono-PEGylated DX-890 with no native protein, di-PEGylated protein, or multi-PEGylated protein. PEG distorts the migration of the PEG-protein through the gel matrix so that the PEG-conjugates appear larger than their predicted molecular masses (˜26 KDa for PEG20-DX-890 and ˜36 KDa for PEG30-DX-890).

### Example 2

### Characterization of DX-890 and PEGylated DX-890

The following PEGylated DX-890 conjugates were tested:

DX-890 conjugated to a 20 kDa (also referred to as “20K”) PEG moiety (conjugated at pH 7.4)

DX-890 conjugated to a 20 kDa PEG moiety (conjugated at pH 6)

DX-890 conjugated to a 30 kDa (also referred to as “30K”) PEG moiety (conjugated at pH 6)

DX-890 conjugated to a 30 kDa PEG moiety (conjugated at pH 7.4)

Determination of Active DX-890 by Functional Quantification (FQ)

DX-890 is an inhibitor of HNE. Determinations of the concentrations of active DX-890 and active PEGylated DX-890 proteins in the stock solutions were performed under conditions of pseudo-irreversible inhibition ([HNE]>>Kᵢ). Under these conditions, inhibition of active HNE by active inhibitor is essentially stoichiometric at a 1:1 molar ratio so that pmoles of active inhibitor present in the reaction is directly determined from the pmoles of active HNE inhibited. Reactions were prepared in which 1.7 nM hNE (˜280×Kᵢ) were incubated in the presence of a range of added volumes of diluted inhibitor stock at 30° C. in 50 mM HEPES, pH 7.5, 150 mM NaCl, and 0.1% Triton X-100. Following a 30 min incubation, substrate was added (to 100 μM) to the enzyme-inhibitor solution and substrate hydrolysis was allowed to proceed at 30° C. for at an additional 30 min. Reactions were stopped by the addition of SDS (to 0.5%) and the final level of fluorescence (λₑₓ=360 nm, λₑₘ=460 nm) was recorded for each reaction. The fluorescence values (F) are converted to pmole of residual uninhibited HNE using Equation 1:

$$HNE ( pmol ) = ( \frac{{F}_{+ DX - 890} - {C}_{- enzyme}}{{F}_{- DX - 890} - {C}_{- enzyme}} ) ( 0.25 pmol HNE ) \qquad Equation 1$$

where:
F_{-DX-890} is the fluorescence observed in the absence of DX-890;
F_{+DX-890} is the fluorescence observed in the presence of DX-890;
C_{-enzyme} is the fluorescence of the No Enzyme Control (0.25 pmol is the amount of enzyme present in the assay)

Active inhibitor present in the reaction is calculated as (total HNE)−(residual free HNE) and this value, corrected for dilutions, is used to calculate the concentration of active inhibitor present in the stock solution.

Ki Measurement

Equilibrium inhibition constants for the PEGylated DX-890 samples were determined according to the tight-binding inhibition model with formation of a reversible complex (1:1 stoichiometry). Reactions were set up with 100 pM enzyme and a range of inhibitor concentrations (0-4 nM) at 30° C. in 50 mM HEPES, pH 7.5, 150 mM NaCl, and 0.1% Triton X-100. Following a 24 h incubation, substrate was added (25 μM) to the enzyme-inhibitor solution and the rate of substrate hydrolysis monitored at an excitation of 360 nm and an emission of 460 nm. Plots of the percent remaining activity versus active inhibitor concentration were fit by nonlinear regression analysis to Equation 1 to determine equilibrium dissociation constants. Both the Dyax DX-890 reference lot 00B16 and the DX-890 used for PEGylations, NBG2016/9 were analyzed for comparison.

$$\% A = 100 - ( \frac{( I + E + {K}_{i} ) - \sqrt{{( I + E + {K}_{i} )}^{2} - 4 \cdot E \cdot I}}{2 \cdot E} ) \cdot 100 \qquad Equation 1$$

Where:
% A=percent activity
I=DX-890
E=HNE concentration
Kᵢ=equilibrium inhibition constant

The Ki's of DX-890 and three of the four PEGylated DX-890 compounds for human neutrophil elastase (HNE) were similar to each other, with the Ki of the 30K PEG DX-890 prepared at pH 7.4 having twice the Ki of native DX-890 (FIG. 1). This result indicates that PEGylation of DX-890 with 20K PEG at pH 6 or 7.4 or with 30K PEG at pH 6 does not affect the potency of DX-890 as an inhibitor of HNE.

### Example 3

### In Vivo Results: Comparison of Ki and PK of DX-890 and PEGylated DX-890

The pharmacokinetics of these three PEGylated DX-890 conjugates with the same Ki as native DX-890 were tested in mice.

Pharmacokinetic Study in Mice:

The pharmacokinetics of DX-890 and the PEGylated DX-890 compounds were measured by iodinating the proteins on available tyrosine residues and measuring their clearance in mice.

Samples were radio-iodinated by the indirect method using the IODO-GEN reagent (method from Pierce Chem. Co., and first described by Chizzonite [J Immunol 147, 1548-1556, 1991; J. Immunol 148, 3117-3124, 1992]). Samples were incubated with the ¹²⁵I-NaI solution for 9 min at which time tyrosine (10 mg/mL, a saturated solution) was added to quench the reaction. After about 15 min a 5 μl aliquot was removed as a standard for counting.

For each labeling reaction, the ¹²⁵I-labeled material (approx. 0.6 mL) was purified using a single 5 mL D-salt 1800 polyacrylamide column (Pierce Chem. Co.). Columns were first washed with 25 mM Tris, 0.4 M NaCl, pH 7.5 containing 2.5% HSA to block nonspecific sites then extensively with the same buffer minus the HSA. Samples were applied in and columns were eluted with a series of 0.3 mL aliquots. Recovery of applied activity in all protein fractions was >75% and the total recovery of applied activity was >90%. The fractions containing peak levels of labeled protein were pooled for animal injections. To prepare the injectate, the pool was diluted with Tris buffer (pH 7.5) so that the 100 μL injection volume contained about 10 μg of labeled material.

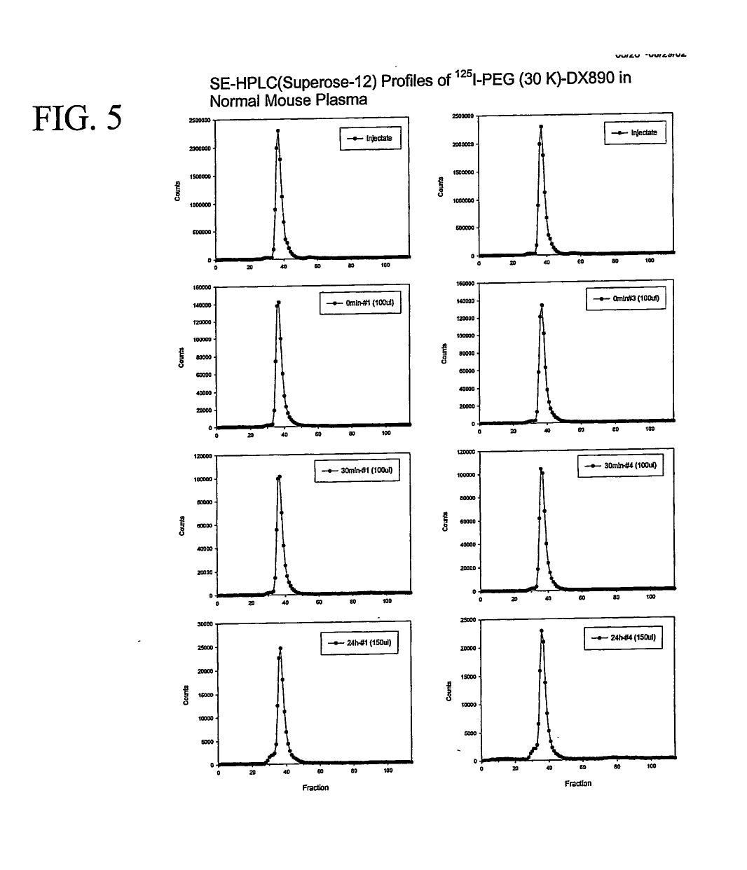

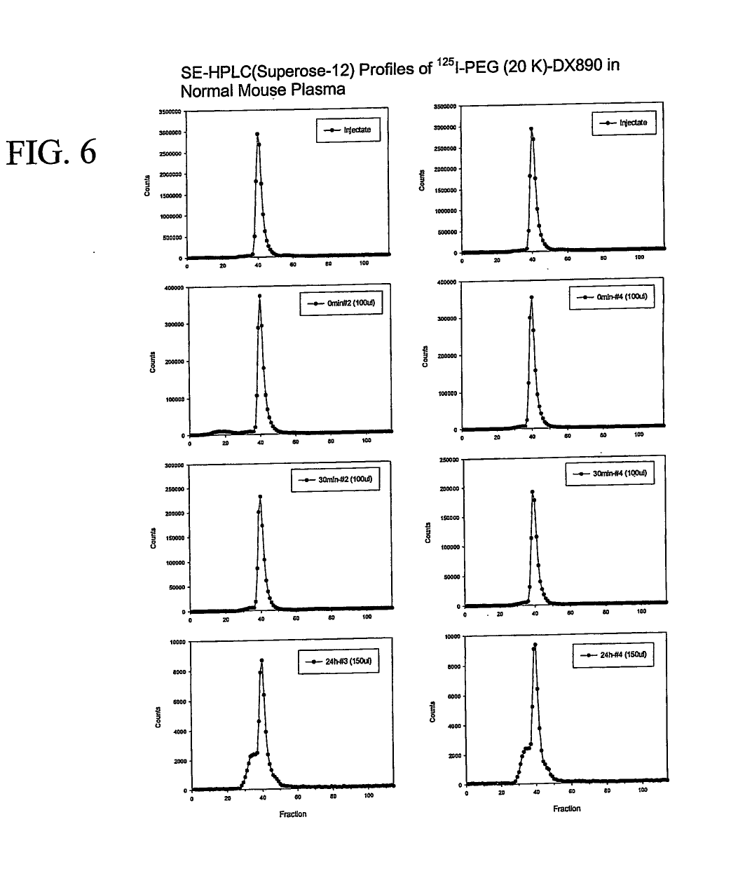

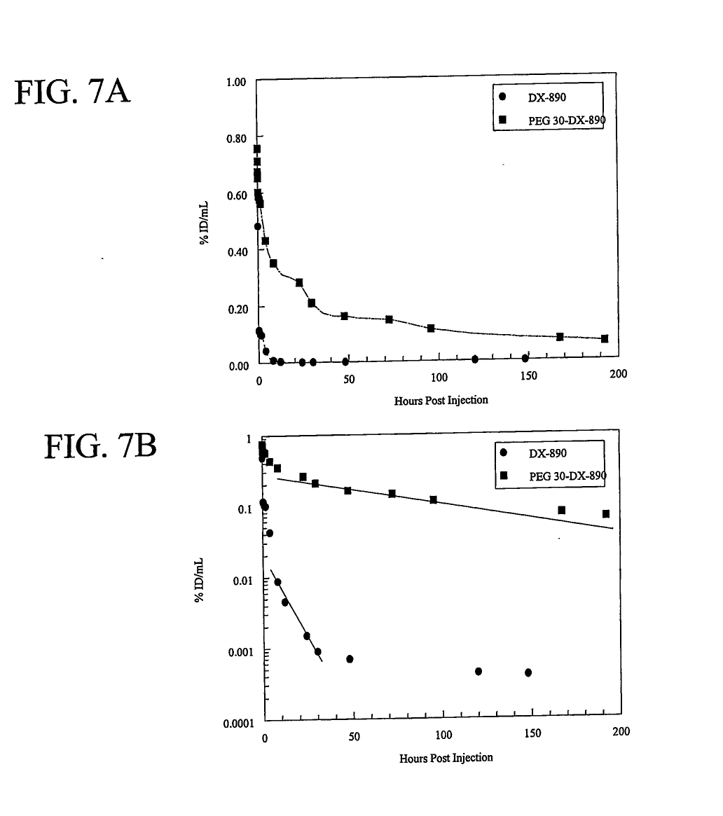

Animals were injected in the tail vein and four animals were sacrificed at approximately 0, 7, 15, 30 and 90 minutes, 4 h, 8 h, 16 h, 24 h after injection, less 4 time points for the native DX-890 because of its likely short half life. Time of injection and time of sampling were recorded. At sacrifice, samples of 0.5 ml were collected into anticoagulant (0.02 ml EDTA). Cells were spun down and separated from plasma, and cells were stored at −20° C. Plasma was divided into two aliquots, one frozen and one stored at 4° C. for immediate analysis. Analysis included gamma counting of all samples. In addition, analysis was performed for two plasma samples (N=2) at each time point, i.e., 0, and 30 minutes, for ¹²⁵I-DX-890, and 0, 30 minutes, and 24 h for the ¹²⁵I-DX-890-PEG conjugates. A SEC-HPLC Superose-12 column was used with an in-line radiation detector (FIGS. 4-7).

The results show that PEGylating DX-890 dramatically improves its beta (elimination) half life by ˜5× (FIGS. 2 and 3). In addition, it appears that the PEGylated DX-890 compounds are more stable in mouse plasma than DX-890 (FIG. 3).

Each set of data shown in FIG. 3 can be fit to a bi-exponential decay curve describing “fast” and “slow” phases of in vivo clearance:
y=Ae^{-αt}+Be^{-βt} Equation 3
Where:
y=Amount of label remaining in plasma at time=t post-administration
A=Total label in “fast” clearance phase
B=Total label in “slow” clearance phase
α=“Fast” clearance phase decay constant
β=“Slow” clearance phase decay constant

t=Time post administration

TABLE 3
Summary of Results from the PK and Ki Measurements in Mice

| | Kᵢ | α Phase | α Phase | β Phase | β Phase Half-life | |
|---|---|---|---|---|---|---|
| Compound | (pM) | Clearance (%) | Half-life (min) | Clearance (%) | (min) | (hr) |
| DX-890 | 7 | 84 | 1.0 | 16 | 79 | 1.3 |
| PEG20-DX-890-6.0 | 7 | 53 | 16.7 | 47 | 296 | 4.9 |
| PEG20-DX-890-7.4 | 8 | 58 | 25.0 | 42 | 307 | 5.1 |
| PEG30-DX-890-6.0 | 8 | 33 | 19.2 | 67 | 505 | 8.4 |

| | | |
|---|---|---|
| PEG30-DX-890-7.4 | 16 | Not Tested in Mice |

The α and β phase decay constants can be converted to half-lives for their respective phases as:

α Phase Half-life = 0.69 (1/α)
β Phase Half-life = 0.69 (1/β)

The percentages of the total label cleared from in vivo circulation through the α and β phases are calculated as:

% α Phase = [A/(A + B)] × 100
% β Phase = [B/(A + B)] × 100

The solid curves through the sets of data plotted in FIG. 3 are four-parameter, least-squares fits of Equation 3 to the data. Table 3 presents the values for % α Phase, α Phase Half-life, % β Phase, and β Phase Half-life extracted from the least squares fits to the in vivo clearance data set obtained for each of the four compounds tested in mice.

Mono-PEGylation of DX-890 with either PEG20 or PEG30 increases in vivo circulating levels of protein in two ways:

1. The half-lives for both α phase and β phase are increased.
Addition of a single 20 KDa PEG moiety at either pH 6.0 or pH 7.4 increases the a phase half life from about 1 min to about 20 min. Mono-PEGylation of DX-890 with 30 KDa PEG at pH 6.0 results in a similar increase in the a phase half-life.
Addition of a single 20 KDa PEG moiety at either pH 6.0 or pH 7.4 increases the β phase half life from about 1 hr to about 5 hr. The β phase half-life is further increased to about 8 hours for the 30 KDa PEG DX-890 derivative.

2. The proportion of labeled protein cleared from in vivo circulation through the slow β phase is increased.
The addition of a single 20 KDa PEG moiety to DX-890 results in an increase in the percentage cleared in the β phase from about 15% for unmodified DX-890 to about 45% for the mono-PEGylated protein.
The addition of a single 30 KDa PEG moiety to DX-890 results in about ⅔ of the clearance occurring through the β phase.

Conclusion: PEGylation with 20K PEG (at pH 6 or 7.4) or with 30K PEG (at pH 6) increases the circulating half life of DX-890 in mice without affecting its potency as an inhibitor of HNE.

TABLE 4
Summary of PK and Ki results

| Preparation | Ki | α half life | β half life |
|---|---|---|---|
| Native DX-890 | 7 pM | 1 min | 79 min |
| +20K PEG, pH 7.4 | 8 pM | 2 min | 5 h 8 min |
| +20K PEG, pH 6 | 7 pM | 15 min | 5 h 1 min |
| +30K PEG, pH 6 | 8 pM | 19 min | 8 h 15 min |
| +30K PEG, pH 7.4 | 16 pM | Not tested | Not tested |

Pharmacokinetic Study in Rabbits
The following PEGylated DX-890 conjugates were tested:
DX-890 conjugated to 20K PEG at pH 7.4
DX-890 conjugated to 20K PEG at pH 6
DX-890 conjugated to 30K PEG at pH 6
DX-890 conjugated to 30K PEG at pH 7.4.

Example 2 described the determination of solution inhibition constants (Ki) for inhibition of hNE by the four PEG-conjugates and for unmodified DX-890. In addition, the report described experiments to measure in vivo clearance properties in mice for unmodified DX-890 along with three of the PEG conjugates (two 20K and one 30K). The results are summarized in Table 4. This example describes the measurement of the pharmacokinetic properties of DX-890 and PEG-30-DX-890 (conjugation at pH 7.4) in rabbits.

Pharmacokinetic properties of DX-890 and PEG-30-DX-890 (conjugation at pH 7.4) were measured by iodinating the proteins and measuring clearance of the radiolabel from circulation in rabbits. The two DX-890 preparations were iodinated with iodine-125 using the iodogen method. After radiolabeling, the two labeled protein preparations were purified from unbound label by size exclusion chromatography (SEC). Fractions from the SEC column having the highest radioactivity were pooled. The purified, radiolabeled preparations were characterized for specific activity by gamma counting and for purity by SEC using a Superose-12 column equipped with an in-line radiation detector.

New Zealand White rabbits (ca. 2.5 Kg) were used for clearance measurements, with one animal used for each of the two labeled protein preparations. The radiolabeled preparation was injected into the animal via an ear vein. One blood sample was collected per animal per time point with early time points at approximately 0, 2.5, 5, 7, 15, 30, 60, and 90 minutes post injection and later time points at 4, 8, 16, 24, 30, 48, 72, 96, 144, 168, and 192 hours post injection. Samples (about 0.5 mL) were collected into anticoagulant (EDTA) containing tubes. Cells were separated from the plasma fraction by centrifugation. The plasma fraction was divided into two aliquots. One plasma aliquot was stored at −70° C. and the other aliquot was kept at 4° C. for immediate analyse's. Sample analyses included radiation counting for clearance rate determinations and SEC chromatography to test for changes in the size distribution of radiolabeled material in vivo (stability).

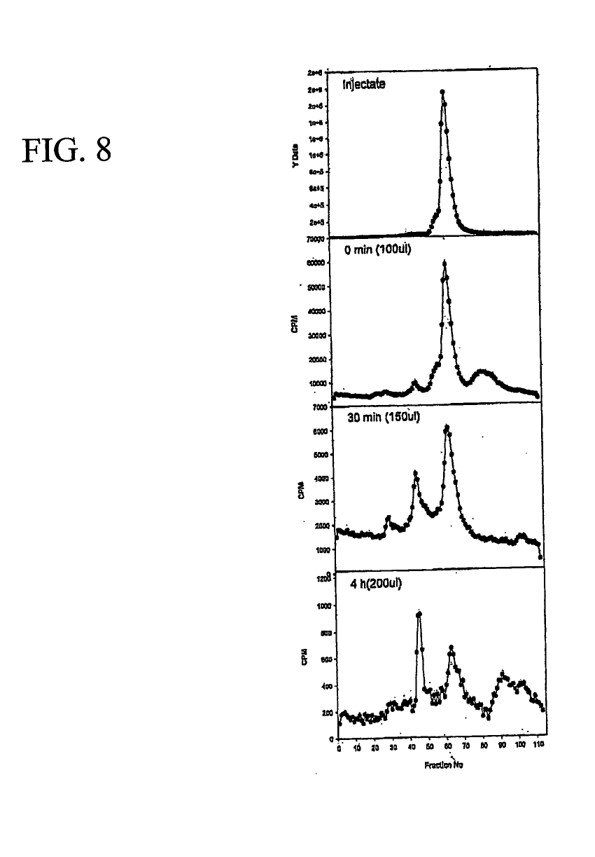

The results of the rabbit clearance study are summarized in FIGS. 8 through 10 and in Table 5.

The PEG-30-DX-890 shows a substantial prolongation of in vivo circulation properties relative to those of the unmodified DX-890. Plasma clearance rates are greatly reduced for the PEGylated protein so that, measured one day post injection, relative levels of circulating radiolabel are more than 100-fold higher for PEG-30-DX-890 than for the unmodified protein (FIG. 8).

A simple bi-exponential fit to the data shows large increases in both the alpha and beta portions of the clearance curve (Table 5). In particular, the value for T_{1/2β} is increased about 25-fold, from about 165 min (2.75 hrs) for the unmodified protein to about 4154 min (˜69 hrs, ˜2.9 days) for PEG-30-DX-890. In addition, the fraction of the total material involved in the slow clearance portion of the curve nearly doubles for the PEGylated protein relative to unmodified DX-890 (Table 5).

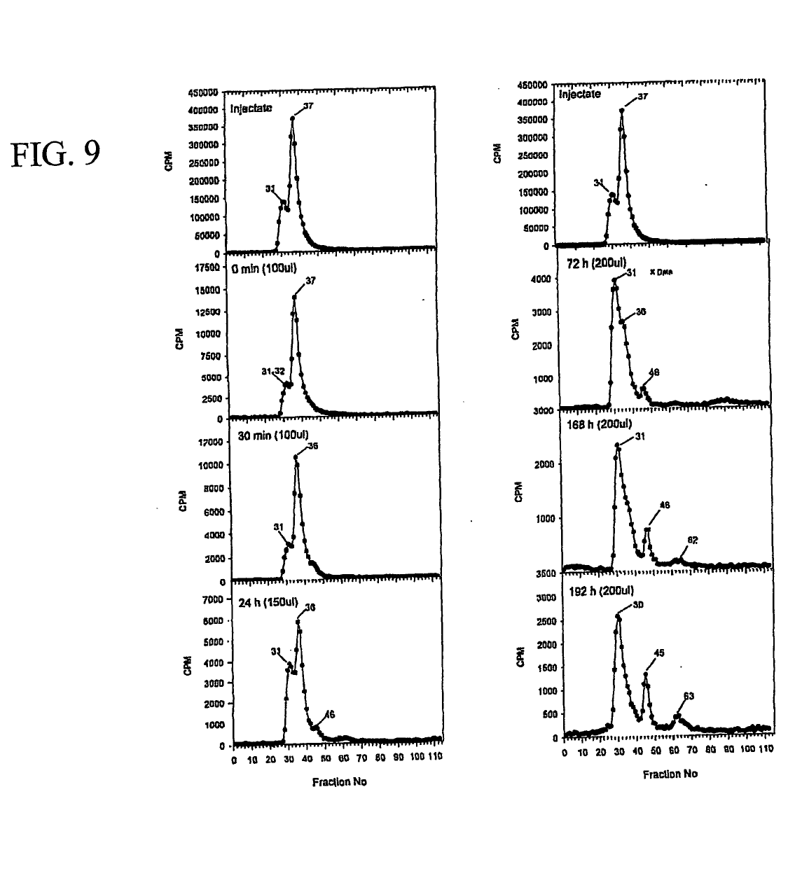

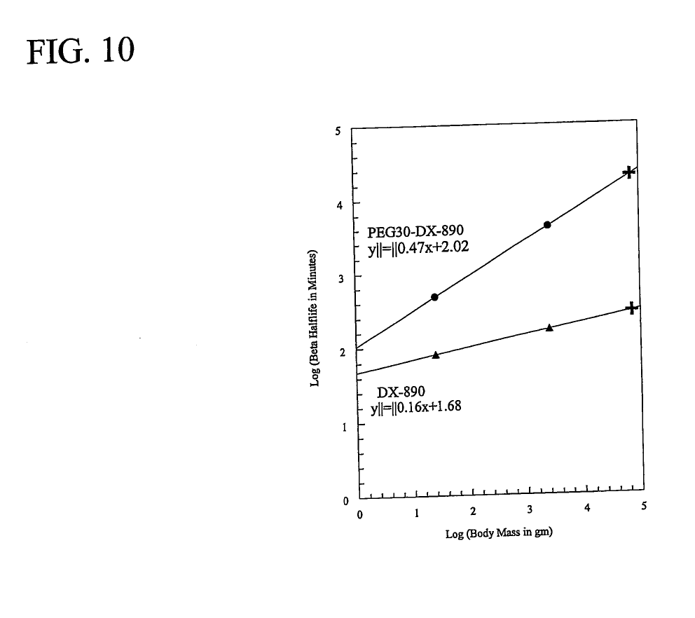

Finally, in vivo stability appears to be improved for the PEGylated protein relative to unmodified DX-890 (FIGS. 9 and 10). SEC analysis of plasma from the rabbit injected with ¹²⁵I-DX-890 (FIG. 9) shows a relatively rapid association of label with higher molecular weight plasma components (earlier eluting peaks). Further, the relative proportion of the total residual circulating label associated with the high molecular weight material increases as time post-injection increases (compare 30 min and 4 hour elution profiles).

In contrast, SEC analyses of plasma samples from the rabbit injected with ¹²⁵I-PEG-30-DX-890 (FIG. 10) shows that almost all of the circulating label is associated with the ¹²⁵I-PEG-30-DX-890 peaks seen in the injectate and that the label remains stably associated with these peaks for at least 72 hours. At later time points (168 hr and 192 hrs), SEC analysis shows that small amounts of label are eluted from the column at the positions (Fractions 45 and 63) similar to those seen in the experiment using unmodified DX-890 (FIG. 9). Label eluting in fraction 63 probably represents unmodified DX-890 (loss of PEG). Label eluting in fraction 45 may reflect unmodified DX-890 in association with high molecular weight serum components (as seen in FIG. 9).

Two peaks are associated with the ¹²⁵I-PEG-30-DX-890 after labeling. It is possible that this behavior on SEC analysis reflects heterogeneity resulting from the PEGylation process. The earlier eluting peak (maximum at fraction 31) appears to clear from circulation at a much slower rate than does the later eluting peak (maximum at fraction 37).

Relative to unmodified DX-890, the PEG-30-DX-890 construct shows substantially prolonged in vivo circulation and stability properties in rabbits. Based on the mouse data, conjugation of either a 20 KDa or 30 KDa PEG moiety to DX-890 results prolongation of in vivo circulation and stability, with the 30 KDa PEG having the greater effect. Conjugation of either 20 KDa PEG or 30 KDa PEG to DX-890 appears to have little effect on the potency of the molecule.

TABLE 5
Clearance Times in Rabbits

| | Dose | | Clearance Times (min) | | | |
|---|---|---|---|---|---|---|
| Compound | μgm | μCi | T_{1/2α} | % α | T_{1/2β} | % β |
| DX-890 | 50 | 83 | 0.4 | 75 | 165 | 25 |
| PEG-30-DX-890 | 46 | 105 | 164 | 55 | 4154 | 45 |

Extrapolation to Larger Mammals

Data on in vivo pharmacokinetic parameters obtained from small laboratory mammals (e.g. rodents, dogs, and monkeys) can be extrapolated to humans by interspecies scaling (summarized by Mahmood and Balian, Life Sciences 7: 579-585, 1996 and Clin Pharmacokinet 36:1-11, 1999). A simple allometric approach describes the relationship between bodyweight (W) and a pharmacokinetic parameter of interest (Y) in terms of the power function:
Y=aW^{b} (Equation 4)
where a and b are the empirically determined coefficient and exponent of the allometric equation, respectively. In general, it has been found that the allometric approach is most effective when more than two species are used. Further, of the three most important pharmacokinetic parameters (total body clearance, volume of distribution, and t_{1/2β}) typically measured, t_{1/2β} is least well predicted by Equation 4. With these caveats in mind, data presented in Tables 2 and 4 can be used to provide crude estimates of expected t_{1/2β} in humans.

FIG. 10 presents data from Tables 2 and 4 plotted as log [Beta Phase Half-Live] vs log [Body Mass] for unmodified DX-890 (triangles) and mono-PEG30-DX-890 (circles). Linear extrapolations of the experimental data for mice (25 gm) and rabbits (2.5 Kg) to humans (70 Kg) are shown by the solid crosses in the figure. The extrapolated values for β-phase half-lives in humans are ˜5 hours for unmodified DX-890 and ˜14 days for mono-PEG30-DX-890. FIG. 10 shows allometric extrapolations to determine β-phase half-lives in humans. Data from mice (see Table 2) and rabbits (see Table 4) are included for unmodified DX-890 (triangles) and mono-PEG30-DX-890 (circles). Linear regressions on the data are shown along with their equations. The values of β-phase half-lives extrapolated to a 70 Kg human are shown by the crosses.

### Example

### Purification of MPEG20K-DX-890 Modified at pH 7.4

Diluted crude PEGylated DX-890 (600 μL), containing 1.3 mg of protein in 5 mM sodium phosphate at pH5.5, was loaded onto a 25 mL SP Sepharose column (Pharmacia). The tri-, di-, mono-PEGylated DX-890, and unPEGylated DX-890 were separated using a gradient of 5 mM sodium phosphate buffer pH 5.5 (Buffer A) and 5 mM sodium phosphate buffer/1M NaCl pH 5.5 (Buffer B) at an approximate flow rate of 1.5 mL per minute.

Fractions containing protein were identified by monitoring absorbance of material exiting the column. Fractions 2, 3, 5-6, 14-15, 17, and 24-25 were collected and concentrated using a Centricon-10 (MW cut off at 10,000 Da) at about 4° C.

The concentrated fractions were analyzed on a SDS-PAGE using 10% gel and tris/glycine as a running buffer. The SDS-PAGE confirmed that fraction 2 contained tri- and di-PEGylated DX-890; fraction 3 contained a mixture of di- and mono-PEGylated protein. Fractions 5-6, 14-15 and 17 contained only mono-PEGylated DX-890. Fractions 24-25 showed no visible bands. This method can be used to prepare preparations of mono-PEGylated DX-890.

### OTHER EMBODIMENTS

A number of embodiments of the invention have been described. Nevertheless, it will be understood that various modifications may be made without departing from the spirit and scope of the invention. Accordingly, other embodiments are within the scope of the following claim.

1. A conjugate comprising DX-890 covalently attached to a poly(alkylene oxide).

2. The conjugate of claim 1, wherein said poly(alkylene oxide) is a poly(ethylene glycol) (PEG).

3. The conjugate of claim 1, wherein the poly(alkylene oxide) is terminally capped with an end-capping moiety selected from the group consisting of hydroxy, alkoxy, substituted alkoxy, alkenoxy, substituted alkenoxy, alkynoxy, substituted alkynoxy, aryloxy and substituted aryloxy.

4. The conjugate of claim 3, wherein the poly(alkylene oxide) is terminally capped with a methoxy group.

5. The conjugate of claim 1, wherein said DX-890 is covalently attached to one or more poly(alkylene oxide) moieties.

6. The conjugate of claim 5, wherein the total poly(alkylene oxide) molecular weight is 20,000 daltons or greater.

7. The conjugate of claim 1, wherein the poly(alkylene oxide) has a structure selected from the group consisting of linear, branched, and forked.

8. The conjugate of claim 1, wherein said DX-890 is covalently attached to said poly(alkylene oxide) by a hydrolyzable linkage.

9. The conjugate of claim 8, wherein said hydrolyzable linkage comprises a collection of atoms selected from the group consisting of carboxylate ester, phosphate ester, hydrolyzable carbamate, anhydride, acetal, ketal, acyloxyalkyl ether, imine, orthoester, thioester, thiolester, and carbonate.

10. The conjugate of claim 9, wherein said hydrolyzable linkage comprises a collection of atoms selected from the group consisting of hydrolyzable carbamates, ester, and carbonate.

11. The conjugate of claim 10, wherein said hyrolyzable linkage is a hydrolyzable carbamate, and said conjugate comprises the structure:
PEG-L-Ar—O—C(O)—NH—P
where
PEG is a poly(ethylene glycol) having a molecular weight of from about 100 to about 60 kD,
L is a hydrolytically stable linking group,
Ar is an aromatic group,
P is DX-890, and
—NH represents an amino group of DX-890.

12. The conjugate of claim 11, wherein PEG is a methoxy PEG possessing the structure CH₃O(CH₂CH₂O)ₙ—CH₂CH₂—, where n ranges from about 10 to about 1200, and L is —O— or —HN—CO—.

13. The conjugate of claim 12, comprising the structure:

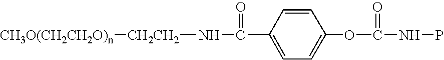

14. The conjugate of claim 1, wherein said DX-890 is covalently attached to said poly(alkylene oxide) by a hydrolytically stable linkage.

15. The conjugate of claim 14, wherein said hydrolytically stable linkage comprises an atom or collection of atoms selected from the group consisting of ether, thioether, amide, and urethane.

16. The conjugate of claim 15, wherein said hydrolytically stable linkage is an amide linkage resulting from covalent attachment of said poly(alkylene oxide) to an amino group of DX-890.

17. The conjugate of claim 16, wherein said poly(alkylene oxide) is covalently attached to one or more amino groups selected from the group consisting of lysine residues and the N-terminus of DX-890.

18. The conjugate of claim 17, wherein said poly(alkylene oxide) is covalently attached to one or more DX-890 amino acid sites selected from the group consisting of Glu¹, lys²⁵, lys²⁷, lys⁴² and lys⁴⁷.

19. The conjugate of claim 1, wherein DX-890 is covalently attached to a single poly(alkylene) oxide moiety.

20. A composition comprising a plurality of mono-PEGylated conjugates of claim 2, wherein each of said mono-PEGylated conjugates comprises a single PEG moiety covalently attached to a different amino acid site of DX-890.

21. The composition of claim 19, further comprising di- and tri-PEGylated DX-890.

22. A composition comprising a conjugate in accordance with claim 1, wherein the composition is substantially free of non-covalently attached poly(alkylene oxide).

23. A purified composition comprising, as its only PEG conjugate component, mono-PEGylated DX-890.

24. A purified composition comprising, as its only PEG conjugate component, di-PEGylated DX-890.

25. A conjugate of claim 1, having a structure selected from either:
P—[NH—CH₂—(CH₂)_{2,3}(OCH₂CH₂)ₙ—OCH₃]₁₋₅
or
P—[NH—C(O)—(CH₂)_{2,3}(OCH₂CH₂)ₙ—OCH₃]₁₋₅
wherein
P is DX-890
—NH represents an amino group of DX-890, and
n ranges from 10 to 1550.

26. A compound that comprises a polypeptide including the amino acid sequence of DX-890 or an amino acid sequence that differs by at least one, but fewer than six amino acid differences from the amino acid sequence of DX-890, wherein the polypeptide is conjugated to a single polyethylene glycol moiety, the polyethylene glycol moiety being at least 18 kDa in molecular weight and attached to the polypeptide to the N-terminus of the polypeptide.

27. The compound of claim 26 wherein the polyethylene glycol moiety is at least 20 kDa in molecular weight.

28. The compound of claim 27 wherein the polyethylene glycol moiety is at least 25 kDa in molecular weight.

29. The compound of claim 26 that inhibits elastase.

30. The compound of claim 26 wherein the polypeptide comprises the amino acid sequence of DX-890.

31. The compound of claim 26 wherein the polypeptide comprises an amino acid sequence that differs by at least one, but fewer than six amino acid differences from the amino acid sequence of DX-890.

32. The compound of claim 31 wherein the polypeptide comprises an amino acid sequence that differs by at least one, but fewer than three amino acid differences from the amino acid sequence of DX-890.

33. The compound of claim 31 wherein the differences are amino acid substitutions.

34. The compound of claim 31 wherein the amino acid sequence is identical to the amino acid sequence of DX-890 at least five positions selected from the group consisting of positions 5, 13, 14, 16, 17, 18, 19, 30, 31, 32, 34, 38, 39, 51, and 55 according to the BPTI numbering.

35. A pharmaceutical preparation that includes (i) a compound according to claim 26, and (ii) a pharmaceutically acceptable carrier.

36. A pharmaceutical preparation that includes (i) a compound according to claim 5, and (ii) a pharmaceutically acceptable carrier.

37. A method of treating or preventing a pulmonary disorder, the method comprising:
administering, to a subject having or at risk for a pulmonary disorder, a compound according to claim 1, in amount effective to treat or prevent the disorder.

38. The method of claim 37 wherein the disorder is cystic fibrosis.

39. The method of claim 37 wherein the disorder is chronic obstructive pulmonary disease.

40. The method of claim 39 wherein the compound is administered in an amount effective to reduce the destructive index in the subject.

41. The method of claim 37 wherein the compound is delivered by inhalation.

42. A method of treating or preventing an inflammatory disorder, the method comprising:
administering, to a subject having or at risk for an inflammatory disorder, a compound according to claim 1, in amount effective to treat or prevent the disorder.

43. The method of claim 42 wherein the disorder is an inflammatory bowel disorder.

44. The method of claim 43 wherein the disorder is Crohn's diseases.

45. The method of claim 43 wherein the disorder is ulcerative colitis.

46. The conjugate of claim 11, wherein said poly(alkylene oxide) is covalently attached to one or more amino groups selected from the group consisting of lysine residues and the N-terminus of DX-890.

47. The composition of claim 20, further comprising di- and tri-PEGylated DX-890.
